(19) United States
(12) Patent Application Publication
(10) Pub. No.: US 2024/0018533 A1
(43) Pub. Date: Jan. 18, 2024

(54) TARGETING MICRORNA TO REGULATE NATIVE GENE FUNCTION BY GENOME EDITING

(71) Applicant: PIONEER HI-BRED INTERNATIONAL, INC., JOHNSTON, IA (US)

(72) Inventors: MARY J FRANK, DES MOINES, IA (US); SHAI LAWIT, URBANDALE, IA (US); JOAN MARIE PHILLIPS, PROLE, IA (US); BO SHEN, JOHNSTON, IA (US); JUN ZHANG, JOHNSTON, IA (US)

(73) Assignee: PIONEER HI-BRED INTERNATIONAL, INC., JOHNSTON, IA (US)

(21) Appl. No.: 17/813,038

(22) PCT Filed: Jan. 19, 2021
(86) PCT No.: PCT/US2021/013863

§ 371 Date: Jul. 17, 2022

Related U.S. Application Data
(60) Provisional application No. 62/963,572, filed on Jan. 21, 2020.

(51) Int. Cl. C12N 15/82; C12N 9/22; C12N 9/78

(52) U.S. Cl. CPC C12N 15/8218; C12N 9/22; C12N 9/78; C12N 2310/141

(57) ABSTRACT

The disclosure provides plants, plant parts, plant cells, seeds and grain containing a targeted genetic modification that inserts an endogenous microRNA recognition sequence into a gene. The disclosure provides plants, plant parts, plant cells, seeds and grain containing a targeted genetic modification that modifies an endogenous microRNA sequence so that the modified microRNA hybridizes to an endogenous gene. Further provided are methods for decreasing expression of a gene of interest by inserting a microRNA recognition sequence into the gene or modifying an endogenous miRNA sequence to hybridize to the gene

### CROSS-REFERENCE TO RELATED APPLICATIONS

This application claims the benefit of U.S. Provisional Application No. 62/963,572 filed on Jan. 21, 2020, which is incorporated herein by reference in its entirety.

### REFERENCE TO SEQUENCE LISTING SUBMITTED ELECTRONICALLY

The official copy of the sequence listing is submitted electronically via EFS-Web as an ASCII formatted sequence listing with a file named “7137-US-PSP_SequenceListing_ST25.txt” created on Jan. 16, 2020 and having a size of 99 kilobytes and is filed concurrently with the specification. The sequence listing contained in this ASCII formatted document is part of the specification and is herein incorporated by reference in its entirety.

### FIELD

This disclosure relates to molecular biology and, specifically, to tissue and/or temporally specific knockdown of target genes.

### BACKGROUND

Gene editing provides a way to precisely insert, knockdown, or modify specific DNA sequences, and has been applied to major crops to modulate gene function and accelerate genetic gain. However, targeted gene knockdown in many cases only generates a recessive, loss-of-function, trait that is lacking tissue and/or temporal specificity.

Therefore, there is a need to develop new compositions and methods for tissue and/or temporal specific targeted gene knockdown. This disclosure provides such compositions and methods.

### SUMMARY

Provided herein are plants, plant parts, plant cells, seeds and grain comprising a targeted genetic modification in a genomic locus of a gene encoding a polypeptide of interest, wherein the targeted genetic modification introduces into the genomic locus an endogenous microRNA (miRNA) recognition sequence, whereby expression of an endogenous miRNA that hybridizes to the endogenous miRNA recognition sequence decreases expression of the polypeptide of interest. In certain embodiments, the miRNA recognition sequence is inserted in the 3′-untranslated region of the gene encoding the polypeptide of interest. In certain embodiments, the miRNA recognition sequence is inserted in the 5′-untranslated region of the gene encoding the polypeptide of interest. In certain embodiments, the miRNA recognition sequence is inserted in the coding region of the gene encoding the polypeptide of interest. In certain embodiments, the endogenous miRNA that hybridizes to the endogenous miRNA recognition sequence comprises the nucleotide sequence of any one of SEQ ID NOs: 1-554. In certain embodiments, the gene encoding the polypeptide of interest encodes a zinc finger containing protein, a kinase, a heat shock protein, a channel protein, an agronomic trait enhancing protein, an insect resistance protein, a disease resistance protein, a herbicide resistance protein, or a protein involved in sterility. In certain embodiments, the gene encoding the polypeptide of interest comprises a nucleic acid sequence that is at least 80% identical to SEQ ID NO: 564.

Further provided are plants, plant parts, plant cells, seeds and grain comprising a targeted genetic modification in the nucleotide sequence of an endogenous microRNA sequence, wherein the targeted genetic modification modifies the endogenous microRNA sequence to encode a modified microRNA that targets a genomic locus of a gene encoding a polypeptide of interest, whereby expression of the modified microRNA decreases expression of the polypeptide of interest. In certain embodiments, the modified miRNA targets a sequence in the 3′-untranslated region of the gene encoding the polypeptide of interest. In certain embodiments, the modified miRNA targets a sequence in the 5′-untranslated region of the gene encoding the polypeptide of interest. In certain embodiments, the modified miRNA targets a sequence in the coding region of the gene encoding the polypeptide of interest. In certain embodiments, the gene encoding the polypeptide of interest encodes a zinc finger containing protein, a kinase, a heat shock protein, a channel protein, an agronomic trait enhancing protein, an insect resistance protein, a disease resistance protein, a herbicide resistance protein, or a protein involved in sterility. In certain embodiments, the gene encoding the polypeptide of interest comprises a nucleic acid sequence that is at least 80% identical to SEQ ID NO: 564. In certain embodiments, the endogenous miRNA sequence comprises the nucleotide sequence of any one of SEQ ID NOs: 1-554.

Provided is a method of altering expression of a polypeptide of interest in a plant cell. In certain embodiments, the method comprises introducing in the plant cell a targeted genetic modification in a genomic locus of a gene encoding the polypeptide of interest, wherein the targeted genetic modification modifies the endogenous gene to encode an endogenous microRNA recognition sequence. In certain embodiments, the method comprises (a) introducing in a regenerable plant cell a targeted genetic modification at a genomic locus of a gene encoding the polypeptide of interest, wherein the targeted genetic modification modifies the genomic locus to encode an endogenous microRNA recognition sequence; and (b) generating the plant, wherein the plant comprises the targeted genetic modification. In certain embodiments, the miRNA recognition sequence is inserted in the 3′-untranslated region of the gene encoding the polypeptide of interest. In certain embodiments, the miRNA recognition sequence is inserted in the 5′-untranslated region of the gene encoding the polypeptide of interest. In certain embodiments, the miRNA recognition sequence is inserted in the coding region of the gene encoding the polypeptide of interest. In certain embodiments, the endogenous miRNA that hybridizes to the endogenous miRNA recognition sequence comprises the nucleotide sequence of any one of SEQ ID NOs: 1-554. In certain embodiments, the gene encoding the polypeptide of interest encodes a zinc finger containing protein, a kinase, a heat shock protein, a channel protein, an agronomic trait enhancing protein, an insect resistance protein, a disease resistance protein, a herbicide resistance protein, or a protein involved in sterility. In certain embodiments, the gene encoding the polypeptide of interest comprises a nucleic acid sequence that is at least 80% identical to SEQ ID NO: 564. In certain embodiments, the targeted genetic modification is introduced using a genome modification technique selected from the group comprising a polynucleotide-guided endonuclease, CRISPR-Cas endonuclease, base editing deaminases, a zinc finger nuclease, a transcription activator-like effector nuclease (TALEN), engineered site-specific meganucleases, or Argonaute.

Further provided is a method of altering expression of a polypeptide of interest in a plant cell. In certain embodiments, the method comprises introducing in the plant cell a targeted genetic modification of an endogenous microRNA to produce a modified microRNA, wherein the modified microRNA targets a gene encoding the polypeptide of interest thereby reducing the expression of the polypeptide of interest. In certain embodiments, the method comprises (a) introducing in a regenerable plant cell a targeted genetic modification in the nucleotide sequence of an endogenous microRNA, wherein the targeted genetic modification modifies the endogenous microRNA encode a modified microRNA that targets a gene encoding the polypeptide of interest; and (b) generating the plant, wherein the plant comprises the targeted genetic modification. In certain embodiments, the modified miRNA targets a sequence in the 3′-untranslated region of the gene encoding the polypeptide of interest. In certain embodiments, the modified miRNA targets a sequence in the 5′-untranslated region of the gene encoding the polypeptide of interest. In certain embodiments, the modified miRNA targets a sequence in the coding region of the gene encoding the polypeptide of interest. In certain embodiments, the gene encoding the polypeptide of interest encodes a zinc finger containing protein, a kinase, a heat shock protein, a channel protein, an agronomic trait enhancing protein, an insect resistance protein, a disease resistance protein, a herbicide resistance protein, or a protein involved in sterility. In certain embodiments, the gene encoding the polypeptide of interest comprises an amino acid sequence that is at least 80% identical to SEQ ID NO: 564. In certain embodiments, the endogenous miRNA sequence comprises the nucleotide sequence of any one of SEQ ID NOs: 1-554. In certain embodiments, the targeted genetic modification is introduced using a genome modification technique selected from the group comprising a polynucleotide-guided endonuclease, CRISPR-Cas endonuclease, base editing deaminases, a zinc finger nuclease, a transcription activator-like effector nuclease (TALEN), engineered site-specific meganucleases, or Argonaute.

### BRIEF DESCRIPTION OF THE DRAWINGS AND THE SEQUENCE LISTING

The disclosure can be more fully understood from the following detailed description and the accompanying drawings and Sequence Listing that form a part of this application, which are incorporated herein by reference.

FIG. 1 provides experimental results showing chlorosis in early leaf tissue from maize culture samples in which the microRNA 156 recognition sequence was inserted into the 3′-untranslated region of the phytoene desaturase gene as compared to a control sample not comprising the recognition sequence.

The sequence listing descriptions summarize the Sequence Listing attached hereto. The Sequence Listing contains one letter codes for nucleotide sequence characters and the single and three letter codes for amino acids as defined in the IUPAC-IUB standards described in Nucleic Acids Research 13:3021-3030 (1985) and in the Biochemical Journal 219(2):345-373 (1984).

TABLE 1
Sequence Listing Description

| SEQ | |
|---|---|
| ID NO: | Description |
| 1-198 | Zea mays miRNA sequences |
| 199-554 | Glycine max miRNA sequences |
| 555 | Zea mays miRNA156B target |
| 556 | Zea mays Phytoene Desaturase (ZM-PDS) 3′UTR in a maize |
| | inbred |
| 557 | Zea mays Phytoene Desaturase CRISPR guide RNA target site 2 |
| | (ZM-PDS-CR2) |
| 558 | HDR oligo template containing miR156B target site |
| | (complementary strand) |
| 559 | Zea mays miR529 target sequence |
| 560 | Zea mays Tasselless 1 (ZM-TSL1) 3′UTR in a maize inbred |
| 561 | Zea mays Tasselless 1 (ZM-TSL1) CRISPR guide RNA site 8 |
| | (ZM-TSL1-CR8) |
| 562 | Zea mays Tasselless 1 (ZM-TSL1) CRISPR guide RNA site 9 |
| | (ZM-TSL1-CR9) |
| 563 | HDR oligo template containing miR156B target site |
| | (complementary strand) for ZM-TSL-CR8 |
| 564 | Zea mays Tasselless 1 (ZM-TSL1) nucleic acid sequence |
| 565 | Zea mays NAC7 (ZM-NAC7) nucleic acid sequence |
| 566 | Zea mays NAC7 (ZM-NAC7) CRISPR guide RNA |

### DETAILED DESCRIPTION

The present disclosure provides plants, plant cells, plant parts, seeds, and/or grain comprising a targeted genetic modification in a genomic locus of a gene of interest, wherein the targeted genetic modification introduces into the genomic locus of the gene of interest an endogenous microRNA recognition sequence, whereby expression of an endogenous microRNA that hybridizes to the microRNA recognition sequence decreases expression of the gene of interest.

A “microRNA recognition sequence,” “miRNA recognition sequence,” “microRNA target sequence,” or the like, as used herein, generally refers to the nucleic acid sequence (e.g., transcribed mRNA) to which a microRNA hybridizes.

The miRNA sequence to which the miRNA recognition sequence hybridizes is not particularly limited and can be any endogenous miRNA sequence of the plant, plant cell, plant part, seed, and/or grain comprising the targeted genetic modification. Representative examples of endogenous miRNA sequences from multiple plants for use in the compositions and methods described herein can be found in the miRbase Sequence Database at miRbase.org.

In certain embodiments, the miRNA sequence is selected from a sequence disclosed in US Patent Application Publication 2016/0017349 or US Patent Application Publication 2008/0115240, each of which are incorporated herein in their entirety by reference.

In certain embodiments the miRNA recognition sequence comprises a nucleic acid sequence that hybridizes to a miRNA sequence that is a least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to a nucleic acid sequence selected from the group SEQ ID NOs: 1-554. In certain embodiments, the miRNA recognition sequence comprises a nucleic acid sequence that hybridizes to a miRNA sequence selected from the group consisting of SEQ ID NOs: 1-554.

As used herein “percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

Unless otherwise stated, sequence identity/similarity values provided herein refer to the value obtained using the BLAST 2.0 suite of programs using default parameters (Altschul, et al., (1997) Nucleic Acids Res. 25:3389-402).

In certain embodiments, expression of the gene of interest is decreased in a targeted location (e.g., a specific tissue) and/or at a certain stage of development and/or under stress conditions (e.g., abiotic stress).

Accordingly, in certain embodiments the selection of the miRNA recognition sequence will depend on the expression pattern of the corresponding endogenous miRNA. For example, to decrease expression of the gene of interest in tassels (e.g., maize tassels) a microRNA recognition sequence that hybridizes to a tassel specific/preferred miRNA, such as, for example miR529 (SEQ ID NO: 198) could be used. miR529 is a tassel preferred microRNA involved with plant reproductive development that has been shown to target squamosa promoter binding protein-like (SBP-box) genes.

Alternatively, to decrease expression of a gene of interest in the roots a microRNA recognition sequence that hybridizes to a root specific/preferred miRNA, such as, for example miR160 (SEQ ID NO: 166) could be used.

To decrease expression of the gene of interest during a plants vegetative stage a microRNA recognition sequence that hybridizes to a miRNA whose expression is upregulated during the vegetative stage such as, for example miR156b (SEQ ID NO: 155) could be used. miR156 is a microRNA which is necessary for the expression of juvenile leaf and shoot development in plants. miR156 regulates the timing of the juvenile-to-adult transition by coordinating expression of multiple pathways in the transition process. miR156 is strongly expressed early in vegetative phase growth, diminishing upon plant transition to adult phase.

Alternatively, to decrease expression of the gene of interest during a plants reproductive stage a microRNA recognition sequence that hybridizes to a miRNA whose expression is upregulated during the reproductive stage such as, for example miR172 (SEQ ID NO: 16) could be used.

As used herein “decrease expression,” “decreased expression,” “knockdown,” and the like are used synonymously and refers to any detectable reduction in the level of the nucleic acid (e.g., mRNA) or protein expression in a sample (e.g., modified plant) as compared to a control sample (e.g., plant not comprising the genome modification). A person of ordinary skill in the art can readily identify a reduction in nucleic acid or protein expression in a sample using routine methods in the art, such as, for example, Western blotting and PCR.

A “genomic locus” as used herein, generally refers to the location on a chromosome of the plant where a gene is found. As used herein, “gene” includes a nucleic acid fragment that expresses a functional molecule such as, but not limited to, a specific protein coding sequence and regulatory elements, such as a promoter, an enhancer, an intron, a 5′-untranslated region (5′-UTR, also known as a leader sequence), or a 3′-untranslated region (3′-UTR). The location of the targeted genetic modification in the genomic locus is not particularly limited, as long as the resulting plant, plant cell, plant part, seed, and/or grain has reduced expression of the gene of interest. In certain embodiments, the targeted genetic modification is in the 3′-UTR of the gene of interest. In certain embodiments, the targeted genetic modification is in the 5′-UTR of the gene of interest. In certain embodiments, the targeted genetic modification is in the coding region of the gene of interest.

An “intron” is an intervening sequence in a gene that is transcribed into RNA but is then excised in the process of generating the mature mRNA. The term is also used for the excised RNA sequences. An “exon” is a portion of the sequence of a gene that is transcribed and is found in the mature messenger RNA derived from the gene but is not necessarily a part of the sequence that encodes the final gene product.

The 5′ untranslated region (5′UTR) (also known as a translational leader sequence or leader RNA) is the region of an mRNA that is directly upstream from the initiation codon. This region is involved in the regulation of translation of a transcript by differing mechanisms in viruses, prokaryotes and eukaryotes.

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

A “targeted genetic modification” refers to the direct modification of any nucleic acid sequence or genetic element by insertion, deletion, or substitution of one or more nucleotides in an endogenous nucleotide sequence. The targeted genetic modification may be introduced using any technique known in the art, such as, for example polynucleotide-guided endonuclease, CRISPR-Cas endonucleases, a transcription activator-like effect nuclease (TALEN), base editing deaminases, zinc finger nuclease, engineered site-specific meganuclease, or Argonaute.

The terms “polypeptide of interest” “gene of interest” and the like are synonymous and generally refer to any polypeptide for which decreased expression is desired.

The gene of interest for use in the methods and compositions described herein is not particularly limited and is reflective of the commercial markets and interests of those involved in the development of the crop. Crops and markets of interest change, and as developing nations open world markets, new crops and technologies will emerge also. In addition, as our understanding of agronomic characteristics and traits such as yield and heterosis increase, the choice of genes for transformation may change accordingly.

General categories of genes of interest include, but are not limited to, those genes involved in information, such as zinc fingers, those involved in communication, such as kinases, those involved in transport, such as porins, and those involved in housekeeping, such as heat shock proteins. More specific categories, for example, include, but are not limited to, genes encoding important traits for agronomics (e.g., yield enhancing, drought resistance, nitrogen use efficiency, maturity, flowering time, senescence, stature, plant architecture, leaf angle and morphology), insect resistance, disease resistance, herbicide resistance, sterility, grain or seed characteristics, and commercial products.

Genes of interest include, generally, those involved in oil, starch, carbohydrate, or nutrient metabolism as well as those affecting seed size, plant development, plant growth regulation, and yield improvement. Plant development and growth regulation also refer to the development and growth regulation of various parts of a plant, such as the flower, seed, root, leaf and shoot.

Other commercially desirable traits are genes and proteins conferring cold, heat, salt, and drought resistance.

Disease and/or insect resistance genes may encode resistance to pests that have great yield drag such as for example, Northern Corn Leaf Blight, head smut, anthracnose, soybean mosaic virus, soybean cyst nematode, root-knot nematode, brown leaf spot, Downy mildew, purple seed stain, seed decay and seedling diseases caused commonly by the fungi—Pythium sp., Phytophthora sp., Rhizoctonia sp., Diaporthe sp. Bacterial blight caused by the bacterium Pseudomonas syringae pv. Glycinea. Genes conferring insect resistance include, for example, Bacillus thuringiensis toxic protein genes (U.S. Pat. Nos. 5,366,892; 5,747,450; 5,737,514; 5,723,756; 5,593,881; and Geiser et al (1986) Gene 48:109); lectins (Van Damme et al. (1994) Plant Mol. Biol. 24:825); and the like.

Herbicide resistance traits may include genes coding for resistance to herbicides that act to inhibit the action of acetolactate synthase (ALS), in particular the sulfonylurea-type herbicides (e.g., the acetolactate synthase ALS gene containing mutations leading to such resistance, in particular the S4 and/or HRA mutations). The ALS-gene mutants encode resistance to the herbicide chlorsulfuron. Glyphosate acetyl transferase (GAT) is an N-acetyltransferase from Bacillus licheniformis that was optimized by gene shuffling for acetylation of the broad-spectrum herbicide, glyphosate, forming the basis of a novel mechanism of glyphosate tolerance in transgenic plants (Castle et al. (2004) Science 304, 1151-1154).

Genes involved in plant growth and development have been identified in plants. One such gene, which is involved in cytokinin biosynthesis, is isopentenyl transferase (IPT). Cytokinin plays a critical role in plant growth and development by stimulating cell division and cell differentiation (Sun et al. (2003), Plant Physiol. 131: 167-176).

In certain embodiments, the polypeptide of interest is a polypeptide that is native to the plant, plant cells, plant parts, seeds, and/or grain (e.g., endogenous gene). In certain embodiments, the polypeptide of interest is a polypeptide that has been inserted into the plant, plant cells, plant parts, seeds, and/or grain, such as, for example, a polypeptide encoded by a gene under the control of a heterologous promoter.

In certain embodiments, the polypeptide of interest is a polypeptide involved in tassel formation and the microRNA recognition sequence comprises a nucleic acid sequence that hybridizes to the nucleic acid sequence of any one of SEQ ID NOs: 1-554.

In certain embodiments, the gene encoding the polypeptide of interest comprises a nucleic acid sequence that is at least 60% (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO: 564 (TLS) and the microRNA recognition sequence comprises a nucleic acid sequence that hybridizes to the nucleic acid sequence of any one of SEQ ID NOs: 1-554.

In certain embodiments, the polypeptide of interest is a polypeptide involved in tassel formation and the microRNA recognition sequence comprises a nucleic acid sequence that hybridizes to the nucleic acid sequence of any one of SEQ ID NOs: 1-197. In certain embodiments, the gene encoding the polypeptide of interest comprises a nucleic sequence that is at least 60% identical to SEQ ID NO: 564 and the microRNA recognition sequence comprises a nucleic acid sequence that hybridizes to the nucleic acid sequence of any one of SEQ ID NOs: 1-197. In certain embodiments, the gene encoding the polypeptide of interest comprises a nucleic acid sequence that is at least 60% identical to SEQ ID NO: 564 (TLS) and the microRNA recognition sequence comprises a nucleic acid sequence that hybridizes the nucleic acid sequence of miR529 (SEQ ID NO: 198) such as, for example SEQ ID NO: 559.

As used herein, the term “plant” includes plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the disclosure, provided that these parts comprise the targeted genetic modification.

Examples of plant species of interest include, but are not limited to, maize (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, conifers, turf grasses (including cool seasonal grasses and warm seasonal grasses).

Vegetables include, for example, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing that which is disclosed include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow cedar (Chamaecyparis nootkatensis), and Poplar and Eucalyptus. In specific embodiments, plants of the present disclosure are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include, for example, grain plants that provide seeds of interest, oil-seed plants, and leguminous plants. Seeds of interest include, for example, grain seeds, such as corn, wheat, barley, rice, sorghum, rye, etc. Oil-seed plants include, for example, cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea.

The present disclosure also provides plants, plant cells, plant parts, seeds, and/or grain comprising a targeted genetic modification of an endogenous microRNA sequence, wherein the targeted genetic modification modifies an endogenous microRNA sequence to encode a modified microRNA sequence that hybridizes to the genomic locus of a gene encoding a polypeptide of interest, thereby decreasing expression of the polypeptide of interest.

As used herein “modified microRNA sequence” “modified miRNA sequence” or the like generally refers to an endogenous microRNA sequence that comprises at least one nucleotide modification, such as an insertion, deletion, and/or substitution. In certain embodiments the modified microRNA is expressed in the same location(s) and/or at the same developmental stage as the corresponding unmodified endogenous microRNA sequence.

The endogenous microRNA sequence to be modified is not particularly limited and can be any of the endogenous microRNA sequence described herein.

In certain embodiments, the endogenous microRNA sequence that is modified comprises a nucleotide sequence of any one of SEQ ID NOs: 1-554, wherein the resulting modified microRNA sequence comprises at least one (e.g., 2, 3, 4, 5, 6, 7, 8, 9, 10, 11, 12, 13, 14, 15, 16, 17, 18, 19, or 20) nucleotide modification as compared to the endogenous microRNA sequence.

In certain embodiments, the modified microRNA is modified to comprise a nucleotide sequence that hybridizes to the genomic locus of the gene of interest and decreases expression of the gene of interest. In certain embodiments, the modified microRNA is modified to comprise a nucleotide sequence that hybridizes under stringent conditions to the genomic locus of the gene of interest and decreases expression of the gene of interest. In certain embodiments, the modified microRNA is modified to comprise a nucleotide sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to a contiguous nucleotide sequence of the genomic locus of the gene of interest and decreases expression of the gene of interest. In certain embodiments, the modified microRNA is modified to comprise a nucleotide sequence that is identical to a contiguous nucleotide sequence of the genomic locus of the gene of interest and decreases expression of the gene of interest.

In certain embodiments, the modified microRNA hybridizes to the protein coding sequence of the gene of interest. In certain embodiments, the modified microRNA hybridizes to a regulatory element of the gene of interest. In certain embodiments, the modified microRNA hybridizes to an intron sequence of the gene of interest. In certain embodiments, the modified microRNA hybridizes to a region of the 5′-UTR of the gene of interest. In certain embodiments, the modified microRNA hybridizes to a region of the 3′-UTR of the gene of interest.

### Methods

Provided herein are methods of decreasing expression of a gene of interest in a plant, plant part, plant cell, seed or grain.

In certain embodiments the method comprises introducing into a plant cell a targeted genetic modification in a genomic locus of a gene of interest, wherein the targeted genetic modification modifies the endogenous gene of interest to encode an endogenous microRNA recognition sequence. In certain embodiments, the plant cell is a regenerable plant cell and the method further comprises generating the plant, wherein the plant comprises the targeted genetic modification. In certain embodiments, the targeted genetic modification is in the 3′-UTR of the gene of interest. In certain embodiments, the targeted genetic modification is in the 5′-UTR of the gene of interest. In certain embodiments, the targeted genetic modification is in the coding region of the gene of interest.

The endogenous microRNA recognition sequence for use in the methods described herein may be any endogenous microRNA recognition sequence described herein. In certain embodiments, the endogenous microRNA recognition sequence comprises a nucleic acid sequence that hybridizes to the nucleic acid sequence of any one of SEQ ID NOs: 1-554.

Also provided is a method of altering expression of a gene of interest in a plant cell comprising introducing in the plant cell a targeted genetic modification in the nucleotide sequence of an endogenous microRNA, wherein the targeted genetic modification modifies the endogenous microRNA to encode a modified microRNA that hybridizes to the gene of interest and decreases expression of the gene of interest.

In certain embodiments, the method comprises introducing in a regenerable plant cell a targeted genetic modification in the nucleotide sequence of an endogenous microRNA, wherein the targeted genetic modification modifies the endogenous microRNA to encode a modified microRNA that targets the gene of interest; and generating the plant, wherein the plant comprises the targeted genetic modification.

The modified microRNA sequence for use in the methods described herein may be any modified microRNA sequence described herein.

Also provided is a method of decreasing the expression of a gene of interest in a tassel of a plant the method comprising introducing into the plant cell a targeted genetic modification in a genomic locus of a gene of interest, wherein the targeted genetic modification modifies the endogenous gene of interest to encode an endogenous microRNA recognition sequence that hybridizes to a tassel specific/preferred microRNA sequence (e.g., miR529, SEQ ID NO: 198).

In certain embodiments, the targeted genetic modification is in the 3′-UTR of the gene of interest. In certain embodiments, the targeted genetic modification is in the 5′-UTR of the gene of interest. In certain embodiments, the targeted genetic modification is in the coding region of the gene of interest.

Further provided is a method of decreasing the expression of a gene of interest during the vegetative stage the method comprising introducing into the plant cell a targeted genetic modification in a genomic locus of a gene of interest, wherein the targeted genetic modification modifies the endogenous gene of interest to encode an endogenous microRNA recognition sequence comprising a nucleic acid sequence that hybridizes to a miRNA sequence whose expression is increased during the vegetative stage (e.g., miR156b SEQ ID NO: 155).

In certain embodiments, the targeted genetic modification is in the 3′-UTR of the gene of interest. In certain embodiments, the targeted genetic modification is in the 5′-UTR of the gene of interest. In certain embodiments, the targeted genetic modification is in the coding region of the gene of interest.

Further provided is a method of decreasing the expression of a gene of interest during the reproductive stage the method comprising introducing into the plant cell a targeted genetic modification in a genomic locus of a gene of interest, wherein the targeted genetic modification modifies the endogenous gene of interest to encode an endogenous microRNA recognition sequence comprising a nucleic acid sequence that hybridizes to a miRNA sequence whose expression is increased during the reproductive stage (e.g., miR172 SEQ ID NO: 16).

In certain embodiments, the targeted genetic modification is in the 3′-UTR of the gene of interest. In certain embodiments, the targeted genetic modification is in the 5′-UTR of the gene of interest. In certain embodiments, the targeted genetic modification is in the coding region of the gene of interest.

As would be understood by a person of ordinary skill in the art, the methods described herein can be modified to decrease expression of a gene of interest in any tissue in which an miRNA is expressed (e.g., root specific decrease), during any development/growth stage in which an miRNA is expressed and/or under any stress condition (e.g., biotic or abiotic stress) in which an miRNA is expressed. In certain embodiments the miRNA recognition sequence is a sequence that hybridizes to a microRNA whose expression level is altered (e.g., increased) in said tissue, developmental stage, or stress condition.

In certain embodiments, the targeted genetic modification is in the 3′-UTR of the gene of interest. In certain embodiments, the targeted genetic modification is in the 5′-UTR of the gene of interest. In certain embodiments, the targeted genetic modification is in the coding region of the gene of interest.

Various methods can be used to introduce the genetic modification at a genomic locus that encodes the gene of interest and/or an endogenous microRNA sequence into the plant, plant part, plant cell, seed, and/or grain. In certain embodiments the targeted genetic modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In some embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, and the like. In some embodiments, the introduction of a DSB can be combined with the introduction of a polynucleotide modification template.

A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease. Tethered DNAs can allow for co-localizing target and template DNA, useful in genome editing and targeted genome regulation, and can also be useful in targeting post-mitotic cells where function of endogenous HR machinery is expected to be highly diminished (Mali et al. 2013 Nature Methods Vol. 10: 957-963.) The polynucleotide modification template may be present transiently in the cell or it can be introduced via a viral replicon.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

In addition to modification by a double strand break technology, modification of one or more bases without such double strand break are achieved using base editing technology, see e.g., Gaudelli et al., (2017) Programmable base editing of A*T to G*C in genomic DNA without DNA cleavage. Nature 551(7681):464-471; Komor et al., (2016) Programmable editing of a target base in genomic DNA without double-stranded DNA cleavage, Nature 533(7603):420-4.

These fusions contain dCas9 or Cas9 nickase and a suitable deaminase, and they can convert e.g., cytosine to uracil without inducing double-strand break of the target DNA. Uracil is then converted to thymine through DNA replication or repair. Improved base editors that have targeting flexibility and specificity are used to edit endogenous locus to create target variations and improve grain yield. Similarly, adenine base editors enable adenine to inosine change, which is then converted to guanine through repair or replication. Thus, targeted base changes i.e., C·G to T·A conversion and A·T to G·C conversion at one more locations made using appropriate site-specific base editors.

In an embodiment, base editing is a genome editing method that enables direct conversion of one base pair to another at a target genomic locus without requiring double-stranded DNA breaks (DSBs), homology-directed repair (HDR) processes, or external donor DNA templates. In an embodiment, base editors include (i) a catalytically impaired CRISPR-Cas9 mutant that are mutated such that one of their nuclease domains cannot make DSBs; (ii) a single-strand-specific cytidine/adenine deaminase that converts C to U or A to G within an appropriate nucleotide window in the single-stranded DNA bubble created by Cas9; (iii) a uracil glycosylase inhibitor (UGI) that impedes uracil excision and downstream processes that decrease base editing efficiency and product purity; and (iv) nickase activity to cleave the non-edited DNA strand, followed by cellular DNA repair processes to replace the G-containing DNA strand.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5- 50, 5-55, 5-60, 5-65, 5- 70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

TAL effector nucleases (TALEN) are a class of sequence-specific nucleases that can be used to make double-strand breaks at specific target sequences in the genome of a plant or other organism. (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H—N—H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as Fokl. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind an 18 nucleotide recognition sequence.

Genome editing using DSB-inducing agents, such as Cas9-gRNA complexes, has been described, for example in U.S. Patent Application US 2015-0082478 A1, published on Mar. 19, 2015, WO2015/026886 A1, published on Feb. 26, 2015, WO2016007347, published on Jan. 14, 2016, and WO201625131, published on Feb. 18, 2016, all of which are incorporated by reference herein.

The term “Cas gene” herein refers to a gene that is generally coupled, associated or close to, or in the vicinity of flanking CRISPR loci in bacterial systems. The terms “Cas gene”, “CRISPR-associated (Cas) gene” are used interchangeably herein. The term “Cas endonuclease” herein refers to a protein encoded by a Cas gene. A Cas endonuclease herein, when in complex with a suitable polynucleotide component, is capable of recognizing, binding to, and optionally nicking or cleaving all or part of a specific DNA target sequence. A Cas endonuclease described herein comprises one or more nuclease domains. Cas endonucleases of the disclosure includes those having a HNH or HNH-like nuclease domain and/or a RuvC or RuvC-like nuclease domain. A Cas endonuclease of the disclosure includes a Cas9 protein, a Cpf1 protein, a C2c1 protein, a C2c2 protein, a C2c3 protein, Cas3, Cas 5, Cas7, Cas8, Cas10, or complexes of these.

As used herein, the terms “guide polynucleotide/Cas endonuclease complex”, “guide polynucleotide/Cas endonuclease system”, “ guide polynucleotide/Cas complex”, “guide polynucleotide/Cas system”, “guided Cas system” are used interchangeably herein and refer to at least one guide polynucleotide and at least one Cas endonuclease that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any of the four known CRISPR systems (Horvath and Barrangou, 2010, Science 327:167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity but can still specifically bind to a DNA target sequence when complexed with a suitable RNA component. (See also U.S. Patent Application US 2015-0082478 A1, published on Mar. 19, 2015 and US 2015-0059010 A1, published on Feb. 26, 2015, both are hereby incorporated in its entirety by reference).

A guide polynucleotide/Cas endonuclease complex can cleave one or both strands of a DNA target sequence. A guide polynucleotide/Cas endonuclease complex that can cleave both strands of a DNA target sequence typically comprise a Cas protein that has all of its endonuclease domains in a functional state (e.g., wild type endonuclease domains or variants thereof retaining some or all activity in each endonuclease domain). Non-limiting examples of Cas9 nickases suitable for use herein are disclosed in U.S. Patent Appl. Publ. No. 2014/0189896, which is incorporated herein by reference.

Other Cas endonuclease systems have been described in PCT patent applications PCT/US16/32073, filed May 12, 2016 and PCT/US16/32028 filed May 12, 2016, both applications incorporated herein by reference.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence. Cas9 protein comprises a RuvC nuclease domain and an HNH (H—N—H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al, Cell 157:1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA.

Any guided endonuclease can be used in the methods disclosed herein. Such endonucleases include, but are not limited to, Cas9 and Cpf1 endonucleases. Many endonucleases have been described to date that can recognize specific PAM sequences (see for example—Jinek et al. (2012) Science 337 p 816-821, PCT patent applications PCT/US16/32073, filed May 12, 2016 and PCT/US16/32028 filed May 12, 2016 and Zetsche B et al. 2015. Cell 163, 1013) and cleave the target DNA at a specific position. It is understood that based on the methods and embodiments described herein utilizing a guided Cas system one can now tailor these methods such that they can utilize any guided endonuclease system.

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide. By “domain” it is meant a contiguous stretch of nucleotides that can be RNA, DNA, and/or RNA-DNA-combination sequence. The VT domain and /or the CER domain of a single guide polynucleotide can comprise a RNA sequence, a DNA sequence, or a RNA-DNA-combination sequence. The single guide polynucleotide being comprised of sequences from the crNucleotide and the tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double strand break) the target site. (See also U.S. Patent Application US 2015-0082478 A1, published on Mar. 19, 2015 and US 2015-0059010 A1, published on Feb. 26, 2015, both are hereby incorporated in its entirety by reference.)

The term “variable targeting domain” or “VT domain” is used interchangeably herein and includes a nucleotide sequence that can hybridize (is complementary) to one strand (nucleotide sequence) of a double strand DNA target site. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site.

The terms “guide RNA/Cas endonuclease complex”, “guide RNA/Cas endonuclease system”, “ guide RNA/Cas complex”, “guide RNA/Cas system”, “gRNA/Cas complex”, “gRNA/Cas system”, “RNA-guided endonuclease” , “RGEN” are used interchangeably herein and refer to at least one RNA component and at least one Cas endonuclease that are capable of forming a complex , wherein said guide RNA/Cas endonuclease complex can direct the Cas endonuclease to a DNA target site, enabling the Cas endonuclease to recognize, bind to, and optionally nick or cleave (introduce a single or double strand break) the DNA target site. A guide RNA/Cas endonuclease complex herein can comprise Cas protein(s) and suitable RNA component(s) of any of the four known CRISPR systems (Horvath and Barrangou, 2010, Science 327:167-170) such as a type I, II, or III CRISPR system. A guide RNA/Cas endonuclease complex can comprise a Type II Cas9 endonuclease and at least one RNA component (e.g., a crRNA and tracrRNA, or a gRNA). (See also U.S. Patent Application US 2015-0082478 A1, published on Mar. 19, 2015 and US 2015-0059010 A1, published on Feb. 26, 2015, both are hereby incorporated in its entirety by reference).

The guide polynucleotide of the methods and compositions described herein may be any polynucleotide sequence that targets the genomic loci of a plant cell comprising a polynucleotide that encodes an amino acid sequence that is at least 90% (e.g., 91%, 92%, 93%, 94%, 95%, 96%, 97%, 98%, 99%, or 100%) identical to a sequence selected from the group consisting of SEQ ID NOs: 9-16. In certain embodiments, the guide polynucleotide is a guide RNA. The guide polynucleotide may also be present in a recombinant DNA construct.

The guide polynucleotide can be introduced into a cell transiently, as single stranded polynucleotide or a double stranded polynucleotide, using any method known in the art such as, but not limited to, particle bombardment, Agrobacterium transformation or topical applications. The guide polynucleotide can also be introduced indirectly into a cell by introducing a recombinant DNA molecule (via methods such as, but not limited to, particle bombardment or Agrobacterium transformation) comprising a heterologous nucleic acid fragment encoding a guide polynucleotide, operably linked to a specific promoter that is capable of transcribing the guide RNA in said cell. The specific promoter can be, but is not limited to, a RNA polymerase III promoter, which allow for transcription of RNA with precisely defined, unmodified, 5′- and 3′-ends (DiCarlo et al., Nucleic Acids Res. 41: 4336-4343; Ma et al., Mol. Ther. Nucleic Acids 3:e161) as described in WO2016025131, published on Feb.18, 2016, incorporated herein in its entirety by reference.

The terms “target site”, “target sequence”, “target site sequence,” target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, or any other DNA molecule in the genome (including chromosomal, choloroplastic, mitochondrial DNA, plasmid DNA) of a cell, at which a guide polynucleotide/Cas endonuclease complex can recognize, bind to, and optionally nick or cleave . The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. Cells include, but are not limited to, human, non-human, animal, bacterial, fungal, insect, yeast, non-conventional yeast, and plant cells as well as plants and seeds produced by the methods described herein. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i) -(iii).

Methods for “modifying a target site” and “altering a target site” are used interchangeably herein and refer to methods for producing an altered target site.

The length of the target DNA sequence (target site) can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other Cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an Cas endonuclease. Assays to measure the single or double-strand break of a target site by an endonuclease are known in the art and generally measure the overall activity and specificity of the agent on DNA substrates containing recognition sites.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

The terms “targeting”, “gene targeting” and “DNA targeting” are used interchangeably herein. DNA targeting herein may be the specific introduction of a knock-out, edit, or knock-in at a particular DNA sequence, such as in a chromosome or plasmid of a cell. In general, DNA targeting can be performed herein by cleaving one or both strands at a specific DNA sequence in a cell with an endonuclease associated with a suitable polynucleotide component. Such DNA cleavage, if a double-strand break (DSB), can prompt NHEJ or HDR processes which can lead to modifications at the target site.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide an guidepolynucleotide/Cas endonuclease complex to a unique DNA target site.

The guide polynucleotide/Cas endonuclease system can be used in combination with a co-delivered polynucleotide modification template to allow for editing (modification) of a genomic nucleotide sequence of interest. (See also U.S. Patent Application US 2015-0082478 A1, published on Mar. 19, 2015 and WO2015/026886 A1, published on Feb. 26, 2015, both are hereby incorporated in its entirety by reference.)

Various methods and compositions can be employed to obtain a cell or organism having a polynucleotide of interest inserted in a target site for a Cas endonuclease. Such methods can employ homologous recombination to provide integration of the polynucleotide of Interest at the target site. In one method provided, a polynucleotide of interest is provided to the organism cell in a donor DNA construct. As used herein, “donor DNA” is a DNA construct that comprises a polynucleotide of Interest to be inserted into the target site of a Cas endonuclease. The donor DNA construct further comprises a first and a second region of homology that flank the polynucleotide of Interest. The first and second regions of homology of the donor DNA share homology to a first and a second genomic region, respectively, present in or flanking the target site of the cell or organism genome. By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5- 50, 5-55, 5-60, 5-65, 5- 70, 5-75, 5-80, 5-5-90, 5-95, 5-100, 5-200, 5-300, 5-400, 5-500, 5-600, 5-700, 5-800, 5-900, 5-1000, 5-1100, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

The amount of sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some embodiments the regions of homology share significant sequence homology to the genomic sequence immediately flanking the target site, it is recognized that the regions of homology can be designed to have sufficient homology to regions that may be further or 3′ to the target site. In still other embodiments, the regions of homology can also have homology with a fragment of the target site along with downstream genomic regions. In one embodiment, the first region of homology further comprises a first fragment of the target site and the second region of homology comprises a second fragment of the target site, wherein the first and second fragments are dissimilar.

As used herein, “homologous recombination” includes the exchange of DNA fragments between two DNA molecules at the sites of homology.

Further uses for guide RNA/Cas endonuclease systems have been described (See U.S. Patent Application US 2015-0082478 A1, published on Mar. 19, 2015, WO2015/026886 A1, published on Feb. 26, 2015, US 2015-0059010 A1, published on Feb. 26, 2015, U.S. application 62/023246, filed on Jul. 7, 2014, and U.S. application 62/036,652, filed on Aug. 13, 2014, all of which are incorporated by reference herein) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Methods for transforming dicots, primarily by use of Agrobacterium tumefaciens, and obtaining transgenic plants have been published, among others, for cotton (U.S. Pat. Nos. 5,004,863, 5,159,135); soybean (U.S. Pat. Nos. 5,569,834, 5,416,011); Brassica (U.S. Pat. No. 5,463,174); peanut (Cheng et al., Plant Cell Rep. 15:653 657 (1996), McKently et al., Plant Cell Rep. 14:699 703 (1995)); papaya (Ling et al., Bio/technology 9:752 758 (1991)); and pea (Grant et al., Plant Cell Rep. 15:254 258 (1995)). For a review of other commonly used methods of plant transformation see Newell, C. A., Mol. Biotechnol. 16:53 65 (2000). One of these methods of transformation uses Agrobacterium rhizogenes (Tepfler, M. and Casse-Delbart, F., Microbiol. Sci. 4:24 28 (1987)). Transformation of soybeans using direct delivery of DNA has been published using PEG fusion (PCT Publication No. WO 92/17598), electroporation (Chowrira et al., Mol. Biotechnol. 3:17 23 (1995); Christou et al., Proc. Natl. Acad. Sci. U.S.A. 84:3962 3966 (1987)), microinjection, or particle bombardment (McCabe et al., Biotechnology 6:923-926 (1988); Christou et al., Plant Physiol. 87:671 674 (1988)).

There are a variety of methods for the regeneration of plants from plant tissues. The particular method of regeneration will depend on the starting plant tissue and the particular plant species to be regenerated. The regeneration, development and cultivation of plants from single plant protoplast transformants or from various transformed explants is well known in the art (Weissbach and Weissbach, Eds.; In Methods for Plant Molecular Biology; Academic Press, Inc.: San Diego, CA, 1988). This regeneration and growth process typically includes the steps of selection of transformed cells, culturing those individualized cells through the usual stages of embryonic development or through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

Terms used in the claims and specification are defined as set forth below unless otherwise specified. It must be noted that, as used in the specification and the appended claims, the singular forms “a,” “an” and “the” include plural referents unless the context clearly dictates otherwise. All cited patents and publications referred to in this application are herein incorporated by reference in their entirety, for all purposes, to the same extent as if each were individually and specifically incorporated by reference.

The following are examples of specific embodiments of some aspects of the invention. The examples are offered for illustrative purposes only and are not intended to limit the scope of the invention in any way.

### EXAMPLE 1

This example demonstrates the introduction of an endogenous microRNA recognition sequence to decrease expression of a gene of interest.

The phytoene desaturase (PDS) gene encodes an essential plant carotenoid biosynthetic enzyme converting 15-cis-phytoene into zeta-carotene. PDS silenced plants display a photobleaching phenotype in leaves. To test whether the down-regulating expression of PDS through microRNA targeting can be achieved through placement of microRNA target site(s) within PDS's expressed transcript the miR156B target site was introduced into the 3′UTR of the PDS gene.

Gene editing via CRISPR-Cas9 was utilized to place the miR156B target site (SEQ ID NO: 555) into the 3′ untranslated region (3′UTR) of the Zea mays PDS gene (SEQ ID NO: 556) in a maize inbred. Guide RNA ZM-PDS-CR2 (SEQ ID NO: 557) created the double-strand break within the maize genome and homology-directed repair (HDR) using a 200-bp oligonucleotide template (SEQ ID NO: 558) inserted the miR156B target site into the maize PDS 3′UTR. The desired gene edit was confirmed by next generation sequencing of samples.

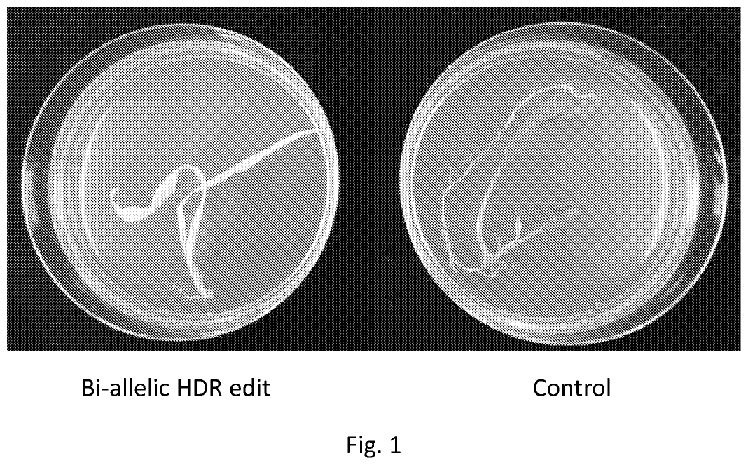

Five tissue cultures samples showed strong chlorosis of early leaf tissue and all were found to have HDR edits containing the miR156B target site on both DNA strands, although not all edits had a perfect HDR matching the template. FIG. 1 provides a representative example showing chlorosis of early leaf tissue in the bi-allelic HDR plants compared to the control non-edited plants. These HDR edited samples rapidly died as anticipated without functional levels of PDS. However, other edited plant seedlings advanced from tissue culture to the greenhouse.

Further sequencing analysis of the edited seedlings advancing to the greenhouse showed that seven plants had one HDR allele with the inserted miR156B target site and one plant had both alleles edited; however, this seedling died after a few days in the greenhouse as expected. It is believed that the plant's early survival was due to an unusually low level of miR156 expression in early tissue culture and vegetative phase allowing for some growth before chlorosis occurred. The other seven identified HDR edited plants had either a wildtype (WT) allele or a second edit involving simple SNPs. All still had one functioning PDS allele without miR156 regulation, allowing for normal plant growth and survival.

Other locations within the PDS transcript were available for gene editing insertion of the miR156B target site, including within the 5′ untranslated region (5′UTR), the coding sequence, and other locations within the 3′UTR. All would be expected to have reduced PDS expression through regulation by miR156. Furthermore, regulation of PDS by other miRNAs such miR172 was considered. miR172 has a complementary expression pattern as compared with miR156. Its expression is highest in mature tissues and lowest in early vegetative tissue. Insertion of the miR172 target site into the PDS transcript would be expected to result in normal growth until adult phase, at which time chlorosis of tissue would be expected.

Taken together, these results demonstrate that the introduction of an endogenous miRNA recognition sequence in a gene of interest results in decreased expression of the gene.

### EXAMPLE 2

The maize tasselless 1 (ZM-TSL1) gene when down-regulated reduces the size and appearance of a maize tassel. Down-regulation of the gene in multiple tissues throughout the plant's growth cycle has negative pleiotropic effects on plant development. Therefore, we tested whether introducing a tassel preferred microRNA recognition sequence in the ZM-TSL1 gene would reduce the tassel size while eliminating other negative effects.

Gene editing via CRISPR-Cas9 was utilized to place the tassel-specific miR529 target site (SEQ ID NO: 559) into the 3′ untranslated region (3′UTR) of the Zea mays TSL1 (SEQ ID NO: 560) in a maize inbred. Guide RNA ZM-TSL1-CR8 (SEQ ID NO: 561) created the double-strand break within the maize genome and homology-directed repair (HDR) using a 200-bp oligonucleotide template (SEQ ID NO: 563) inserted the miR529 target site into the maize TSL1 3′UTR. The template was designed to create as few alterations as possible when compared to the endogenous ZM-TSL1 sequence while allowing for the presence of the 21 bp miR529 target site within the 3′UTR. The design also altered one base in the PAM motif within the template in order to prevent further double stranded breaks within any edited plants. The desired gene edit was confirmed in twenty T0 seedlings by next generation sequencing of samples. Fifteen of those samples set seed, with resulting progeny still to be analyzed and phenotyped.

Other locations within the ZM-TSL1 transcript were available for insertion of the miR529 target site by gene editing, including within the 5′ untranslated region (5′UTR), the coding sequence, and other locations within the 3′UTR. For example, guide RNA ZM-TLS1-CR9 (SEQ ID NO: 562) is also available within the 3′UTR providing a guide RNA site for miR529 target site insertion. Any miR529 target site insertions within the expressed ZM-TSL1 gene regardless of location would be expected to reduce TSL1 expression in the tassel without affecting ear growth.

### EXAMPLE 3

The maize NAC7 (ZM-NAC7) gene is a novel QTL controlling functional stay-green that was discovered in a mapping population derived from the Illinois High Protein 1 (IHP1) and Illinois Low Protein 1 (ILP1) lines, which show very different rates of leaf senescence. Transgenic maize lines where ZM-NAC7 was down-regulated by RNAi showed delayed senescence and increased both biomass and nitrogen accumulation in vegetative tissues, demonstrating that NAC7 functions as a negative regulator of the stay-green trait (J Zhang, et al, Plant Biotechnol J. 2019 17(12):2272-2285). This example demonstrates utilizing the miR156e recognition sequence to regulate expression of endogenous ZM-NAC7.

During early development in Arabidopsis, expression of miR156 is initially high and then steadily decreases as the plant matures (G Wu, et al, Cell, 2009, 138 (4): p750-759). Therefore, the insertion of the miRNA156 recognition sequence into the 3′ UTR of ZM-NAC7 should reduce the expression of ZM-NAC7 in the vegetative stage and increase photosynthesis, while maintaining certain endogenous ZM-NAC7 expression in the late developmental stage of maize to accelerate senescence and dry down grains.

To insert the miRNA156e recognition sequence into ZM-NAC7, a guide RNA (SEQ ID NO: 566) was designed to target a sequence in the 3′-UTR of the ZM-NAC7 gene (SEQ ID NO: 565) in a maize inbred. The single guide RNA will create the double-strand break in ZM-NAC7 genomic DNA. Homology-directed repair using an oligonucleotide template containing the miR156e recognition sequence will insert the target site for miR156e (SEQ ID NO: 63). The desired gene edit will be confirmed by next generation sequencing of samples. Positive samples will be analyzed and phenotyped.

1. A plant cell comprising a targeted genetic modification in a genomic locus of a gene encoding a polypeptide of interest, wherein the targeted genetic modification introduces into the genomic locus an endogenous microRNA recognition sequence, whereby expression of an endogenous microRNA that hybridizes to the endogenous microRNA recognition sequence decreases expression of the polypeptide of interest.

2. The plant cell of claim 1, wherein the microRNA recognition sequence is inserted into the 3′ untranslated region of the gene encoding the polypeptide of interest.

3. The plant cell of claim 1, wherein the microRNA recognition sequence is inserted into the 5′ untranslated region of the gene encoding the polypeptide of interest.

4. The plant cell of claim 1, wherein the microRNA recognition sequence is inserted into the coding region of the gene encoding the polypeptide of interest.

5. The plant cell of claim 1, wherein the endogenous miRNA that hybridizes to the endogenous miRNA recognition sequence comprises the nucleotide sequence of any one of SEQ ID NOs: 1-554.

6. The plant cell of claim 1, wherein the gene encoding the polypeptide of interest encodes a zinc finger containing protein, a kinase, a heat shock protein, a channel protein, an agronomic trait enhancing protein, an insect resistance protein, a disease resistance protein, a herbicide resistance protein, or a protein involved in sterility.

7. The plant cell of claim 6 wherein the gene encoding the polypeptide of interest comprises a nucleic acid sequence that is at least 80% identical to SEQ ID NO: 564.

8. A plant comprising the plant cell of claim 1.

9. A plant cell comprising a targeted genetic modification in the nucleotide sequence of an endogenous microRNA sequence, wherein the targeted genetic modification modifies the endogenous microRNA sequence to encode a modified microRNA that targets a genomic locus of a gene encoding a polypeptide of interest, whereby expression of the modified microRNA decreases expression of the polypeptide of interest.

10. The plant cell of claim 9, wherein the modified microRNA targets a sequence in the 3′ untranslated region of the gene encoding the polypeptide of interest.

11. The plant cell of claim 9, wherein the modified microRNA targets a sequence in the 5′ untranslated region of the encoding the polypeptide of interest.

12. The plant cell of claim 9, wherein the modified microRNA targets a sequence in the coding region of the gene encoding the polypeptide of interest.

13. The plant cell of claim 9, wherein the gene encoding the polypeptide of interest encodes a zinc finger containing protein, a kinase, a heat shock protein, a channel protein, an agronomic trait enhancing protein, an insect resistance protein, a disease resistance protein, a herbicide resistance protein, or a protein involved in sterility.

14. The plant cell of claim 13 wherein the gene encoding the polypeptide of interest comprises a nucleic acid sequence that is at least 80% identical to SEQ ID NO: 564.

15. The plant cell of claim 9, wherein the endogenous miRNA sequence comprises the nucleotide sequence of any one of SEQ ID NOs: 1-554.

16. A plant comprising the plant cell of claim 9.

17-25. (canceled)

26. A method of producing a plant having decreased expression of a polypeptide of interest, the method comprising:
(a) introducing in a regenerable plant cell a targeted genetic modification at a genomic locus of a gene encoding the polypeptide of interest, wherein the targeted genetic modification modifies the genomic locus to encode an endogenous microRNA recognition sequence; and
(b) generating the plant, wherein the plant comprises the targeted genetic modification.

27-29. (canceled)

30. The method of claim 26, wherein the endogenous miRNA recognition sequence comprises a nucleotide sequence that hybridizes to the nucleotide sequence of any one of SEQ ID NOs: 1-554.

31. The method of claim 26, wherein the gene encoding the polypeptide of interest encodes a zinc finger containing protein, a kinase, a heat shock protein, a channel protein, an agronomic trait enhancing protein, an insect resistance protein, a disease resistance protein, a herbicide resistance protein, or a protein involved in sterility.

32. (canceled)

33. The method of claim 26, wherein the targeted genetic modification is introduced using a genome modification technique selected from the group comprising a polynucleotide-guided endonuclease, CRISPR-Cas endonuclease, or base editing deaminases.

34-49. (canceled)


SEQUENCE LISTING (566 sequences)

SEQ ID NO: 1 - DNA, 22 bp, Zea mays
aaaatgggagcagagcaggttt

SEQ ID NO: 2 - DNA, 19 bp, Zea mays
aaacctggctctgatacca

SEQ ID NO: 3 - DNA, 20 bp, Zea mays
aaagaagaagaagaagaaga

SEQ ID NO: 4 - DNA, 21 bp, Zea mays
aacaataggaatgggaggcat

SEQ ID NO: 5 - DNA, 21 bp, Zea mays
aacacatgtggattgaggtca

SEQ ID NO: 6 - DNA, 18 bp, Zea mays
aacagtctccttggctga

SEQ ID NO: 7 - DNA, 20 bp, Zea mays
aaccaggctctgataccaga

SEQ ID NO: 8 - DNA, 19 bp, Zea mays
aagatctgtggcgccgaac

SEQ ID NO: 9 - DNA, 19 bp, Zea mays
aagatctgtggcgccgacc

SEQ ID NO: 10 - DNA, 19 bp, Zea mays
aagatctgtggcgccgaga

SEQ ID NO: 11 - DNA, 19 bp, Zea mays
aagatctgtggcgccgagc

SEQ ID NO: 12 - DNA, 19 bp, Zea mays
aagatctgtggcgccgagt

SEQ ID NO: 13 - DNA, 21 bp, Zea mays
aagctcaggagggatagcgcc

SEQ ID NO: 14 - DNA, 19 bp, Zea mays
acgatctgtggcgccgagc

SEQ ID NO: 15 - DNA, 19 bp, Zea mays
agaataaaaataataatat

SEQ ID NO: 16 - DNA, 20 bp, Zea mays
agaatcttgatgatgctgca

SEQ ID NO: 17 - DNA, 20 bp, Zea mays
agactacaattatctgatca

SEQ ID NO: 18 - DNA, 20 bp, Zea mays
agagaagaagaagaagaagg

SEQ ID NO: 19 - DNA, 21 bp, Zea mays
agagcgtccttcagtccactc

SEQ ID NO: 20 - DNA, 21 bp, Zea mays
agagttggaggaaagcaaacc

SEQ ID NO: 21 - DNA, 18 bp, Zea mays
agcagtctccttggcttc

SEQ ID NO: 22 - DNA, 20 bp, Zea mays
agccaaggatgacttgccga

SEQ ID NO: 23 - DNA, 20 bp, Zea mays
agccaaggatgacttgccgg

SEQ ID NO: 24 - DNA, 20 bp, Zea mays
agccaaggatgacttgccta

SEQ ID NO: 25 - DNA, 20 bp, Zea mays
agccaaggatgacttgcctg

SEQ ID NO: 26 - DNA, 21 bp, Zea mays
aggaagaaccggtaataagca

SEQ ID NO: 27 - DNA, 21 bp, Zea mays
aggattagagggacttgaacc

SEQ ID NO: 28 - DNA, 21 bp, Zea mays
aggcagtggcttggttaaggg

SEQ ID NO: 29 - DNA, 21 bp, Zea mays
atagccaaggatgacttgcct

SEQ ID NO: 30 - DNA, 19 bp, Zea mays
atgatctgtggcgccgagc

SEQ ID NO: 31 - DNA, 22 bp, Zea mays
atggaatatatgacaaaggtgg

SEQ ID NO: 32 - DNA, 24 bp, Zea mays
atggttcaagaaagcccatggaaa

SEQ ID NO: 33 - DNA, 19 bp, Zea mays
caacctggctctgatacaa

SEQ ID NO: 34 - DNA, 19 bp, Zea mays
caacctggctctgatacca

SEQ ID NO: 35 - DNA, 19 bp, Zea mays
caacctggctctgataccc

SEQ ID NO: 36 - DNA, 20 bp, Zea mays
caagaagaagaagaagaaga

SEQ ID NO: 37 - DNA, 20 bp, Zea mays
caagaagaagaagaagaagg

SEQ ID NO: 38 - DNA, 20 bp, Zea mays
caccaggctctgataccaat

SEQ ID NO: 39 - DNA, 21 bp, Zea mays
cacgtgcgctccttctccaac

SEQ ID NO: 40 - DNA, 21 bp, Zea mays
cacgtgcgctccttctccagc

SEQ ID NO: 41 - DNA, 21 bp, Zea mays
cacgtgctccccttctccacc

SEQ ID NO: 42 - DNA, 20 bp, Zea mays
cacgtggtctccttctccat

SEQ ID NO: 43 - DNA, 19 bp, Zea mays
cagatctgtggcgccgacc

SEQ ID NO: 44 - DNA, 19 bp, Zea mays
cagatctgtggcgccgagc

SEQ ID NO: 45 - DNA, 21 bp, Zea mays
cagccaaggatgacttgccgg

SEQ ID NO: 46 - DNA, 21 bp, Zea mays
cagctcctgcagcatctgttc

SEQ ID NO: 47 - DNA, 18 bp, Zea mays
caggggtgtgggacccac

SEQ ID NO: 48 - DNA, 18 bp, Zea mays
caggggtgtgggacccca

SEQ ID NO: 49 - DNA, 18 bp, Zea mays
caggggtgtgggacccca

SEQ ID NO: 50 - DNA, 21 bp, Zea mays
ccagcgctgcactcaattacg

SEQ ID NO: 51 - DNA, 18 bp, Zea mays
cccagtctccttggctac

SEQ ID NO: 52 - DNA, 20 bp, Zea mays
cccgccttgcaccaagtgaa

SEQ ID NO: 53 - DNA, 19 bp, Zea mays
ccggagatgaaggagccga

SEQ ID NO: 54 - DNA, 21 bp, Zea mays
cctgtgcctgcctcttccatt

SEQ ID NO: 55 - DNA, 21 bp, Zea mays
cgctatctatcctgagctcca

SEQ ID NO: 56 - DNA, 19 bp, Zea mays
cgggagatgaaggagccat

SEQ ID NO: 57 - DNA, 19 bp, Zea mays
ctacctggctctgatacca

SEQ ID NO: 58 - DNA, 22 bp, Zea mays
ctattccccagcggagtcgcca

SEQ ID NO: 59 - DNA, 21 bp, Zea mays
ctcaggagagatgacaccgcg

SEQ ID NO: 60 - DNA, 18 bp, Zea mays
ctcagtctccttggctga

SEQ ID NO: 61 - DNA, 19 bp, Zea mays
ctcagtctctttggctata

SEQ ID NO: 62 - DNA, 22 bp, Zea mays
ctccaaagggatcgcattgatc

SEQ ID NO: 63 - DNA, 21 bp, Zea mays
ctgacagaagagagtgagcac

SEQ ID NO: 64 - DNA, 21 bp, Zea mays
ctgcactgcctcttccctggc

SEQ ID NO: 65 - DNA, 19 bp, Zea mays
ctggagatgaaggagccgg

SEQ ID NO: 66 - DNA, 20 bp, Zea mays
cttggattgaagggagctcc

SEQ ID NO: 67 - DNA, 22 bp, Zea mays
gaaatgggagcagagcaggttt

SEQ ID NO: 68 - DNA, 19 bp, Zea mays
gaacctggctctgatacca

SEQ ID NO: 69 - DNA, 20 bp, Zea mays
gaagaagaagaagaagaaac

SEQ ID NO: 70 - DNA, 20 bp, Zea mays
gaagaagaagaagaagaaga

SEQ ID NO: 71 - DNA, 20 bp, Zea mays
gaagaagaagaagaagaagc

SEQ ID NO: 72 - DNA, 21 bp, Zea mays
gaagctgccagcatgatctaa

SEQ ID NO: 73 - DNA, 21 bp, Zea mays
gaagctgccagcatgatctag

SEQ ID NO: 74 - DNA, 21 bp, Zea mays
gaagctgccagcatgatctga

SEQ ID NO: 75 - DNA, 21 bp, Zea mays
gaagctgccagcatgatctgg

SEQ ID NO: 76 - DNA, 20 bp, Zea mays
gaatcttgatgatgctgcat

SEQ ID NO: 77 - DNA, 19 bp, Zea mays
gagatctgtggcgccgagc

SEQ ID NO: 78 - DNA, 21 bp, Zea mays
gagctcccttcgatccaatcc

SEQ ID NO: 79 - DNA, 20 bp, Zea mays
gagctctcttcagtccactc

SEQ ID NO: 80 - DNA, 21 bp, Zea mays
gaggtgagccgagccaatatc

SEQ ID NO: 81 - DNA, 22 bp, Zea mays
gatcatgctgtgcagtttcatc

SEQ ID NO: 82 - DNA, 19 bp, Zea mays
gatcatgttgcagcttcat

SEQ ID NO: 83 - DNA, 22 bp, Zea mays
gatcgtgctgcgcagtttcacc

SEQ ID NO: 84 - DNA, 21 bp, Zea mays
gcagccaaggatgacttgccg

SEQ ID NO: 85 - DNA, 21 bp, Zea mays
gcctggctccctgtatgccac

SEQ ID NO: 86 - DNA, 21 bp, Zea mays
gcctggctccctgtatgccgc

SEQ ID NO: 87 - DNA, 21 bp, Zea mays
gcgtgcatggtgccaagcata

SEQ ID NO: 88 - DNA, 21 bp, Zea mays
gctcaccctctatctgtcagt

SEQ ID NO: 89 - DNA, 22 bp, Zea mays
gctcactgctctatctgtcacc

SEQ ID NO: 90 - DNA, 22 bp, Zea mays
gctcactgctctatctgtcatc

SEQ ID NO: 91 - DNA, 22 bp, Zea mays
gctcactgctctttctgtcatc

SEQ ID NO: 92 - DNA, 21 bp, Zea mays
ctcacttctctctctgtcagt

SEQ ID NO: 93 - DNA, 19 bp, Zea mays
gctcatgttgcagcttcaa

SEQ ID NO: 94 - DNA, 22 bp, Zea mays
gctcgcttctctttctgtcagc

SEQ ID NO: 95 - DNA, 21 bp, Zea mays
gctgtaccctctctcttcttc

SEQ ID NO: 96 - DNA, 19 bp, Zea mays
ggacagtctcaggtagaca

SEQ ID NO: 97 - DNA, 24 bp, Zea mays
ggacccaacgcgactgacggataa

SEQ ID NO: 98 - DNA, 21 bp, Zea mays
ggatattggtgcggttcaatc

SEQ ID NO: 99 - DNA, 21 bp, Zea mays
ggattgagccgcgtcaatatc

SEQ ID NO: 100 - DNA, 21 bp, Zea mays
ggcaagtcatctggggctacg

SEQ ID NO: 101 - DNA, 19 bp, Zea mays
ggcaggccttctggctaag

SEQ ID NO: 102 - DNA, 20 bp, Zea mays
ggcaggtcttcttggctagc

SEQ ID NO: 103 - DNA, 18 bp, Zea mays
ggcagtctccttggctag

SEQ ID NO: 104 - DNA, 18 bp, Zea mays
ggcagtctccttggctga

SEQ ID NO: 105 - DNA, 23 bp, Zea mays
ggcatcgggggcgcaacgcccct

SEQ ID NO: 106 - DNA, 20 bp, Zea mays
ggccgggggacggaccagga

SEQ ID NO: 107 - DNA, 20 bp, Zea mays
ggccgggggacggaccggga

SEQ ID NO: 108 - DNA, 20 bp, Zea mays
ggccgggggacggatcggga

SEQ ID NO: 109 - DNA, 20 bp, Zea mays
ggccgggggacggcccggga

SEQ ID NO: 110 - DNA, 21 bp, Zea mays
gggcaaccccccgttggcagg

SEQ ID NO: 111 - DNA, 21 bp, Zea mays
gggcaaccccccgttggcagg

SEQ ID NO: 112 - DNA, 21 bp, Zea mays
gggcgcagtggtttatcgatc

SEQ ID NO: 113 - DNA, 21 bp, Zea mays
gggcttctctttcttggcagg

SEQ ID NO: 114 - DNA, 21 bp, Zea mays
gggcttggtgcagctcgggaa

SEQ ID NO: 115 - DNA, 21 bp, Zea mays
ggggcggactgggaacacatg

SEQ ID NO: 116 - DNA, 19 bp, Zea mays
ggggggggggggggaaaaa

SEQ ID NO: 117 - DNA, 22 bp, Zea mays
gggtgtcatctcgcctgaagca

SEQ ID NO: 118 - DNA, 22 bp, Zea mays
gggtgtcatctcgcctgaagca

SEQ ID NO: 119 - DNA, 23 bp, Zea mays
ggtcatgctgctgcagcctcact

SEQ ID NO: 120 - DNA, 20 bp, Zea mays
ggtcatgctgtagtttcatc

SEQ ID NO: 121 - DNA, 21 bp, Zea mays
gtagccaaggatgacttgcct

SEQ ID NO: 122 - DNA, 22 bp, Zea mays
gtcagtgcaatccctttggaat

SEQ ID NO: 123 - DNA, 21 bp, Zea mays
gtgaagtgtttgggggaactc

SEQ ID NO: 124 - DNA, 22 bp, Zea mays
gtgaagtgtttgggggaactct

SEQ ID NO: 125 - DNA, 21 bp, Zea mays
gtgagccgaaccaatatcact

SEQ ID NO: 126 - DNA, 21 bp, Zea mays
gtgcagttctcctctggcacg

SEQ ID NO: 127 - DNA, 21 bp, Zea mays
gtgcggctctcctctggcatg

SEQ ID NO: 128 - DNA, 21 bp, Zea mays
gtgctcccttcaaaccaataa

SEQ ID NO: 129 - DNA, 21 bp, Zea mays
gtgctcccttcacaccaataa

SEQ ID NO: 130 - DNA, 19 bp, Zea mays
gtggagatgaaggagccga

SEQ ID NO: 131 - DNA, 21 bp, Zea mays
gtgtggctctcctctggcatg

SEQ ID NO: 132 - DNA, 21 bp, Zea mays
gttcaataaagctgtgggaaa

SEQ ID NO: 133 - DNA, 22 bp, Zea mays
gttcccttcaagcacttcacat

SEQ ID NO: 134 - DNA, 22 bp, Zea mays
gttccttccaaacacttcacca

SEQ ID NO: 135 - DNA, 22 bp, Zea mays
gttctatgcaagcacttcacga

SEQ ID NO: 136 - DNA, 22 bp, Zea mays
gttctcttcaagcacttcacga

SEQ ID NO: 137 - DNA, 21 bp, Zea mays
gttggtgatctcggaccaggc

SEQ ID NO: 138 - DNA, 19 bp, Zea mays
tagatctgtggcgccgagt

SEQ ID NO: 139 - DNA, 21 bp, Zea mays
tagccaagaatggcttgccta

SEQ ID NO: 140 - DNA, 21 bp, Zea mays
tagccagggatgatttgcctg

SEQ ID NO: 141 - DNA, 19 bp, Zea mays
tatcatgttgcagcttctc

SEQ ID NO: 142 - DNA, 21 bp, Zea mays
tattgacgcggttcaattcga

SEQ ID NO: 143 - DNA, 21 bp, Zea mays
tattggtgaggttcaatccga

SEQ ID NO: 144 - DNA, 21 bp, Zea mays
tcattgagcgcagcgttgatg

SEQ ID NO: 145 - DNA, 22 bp, Zea mays
tccaaagggatcgcattgatcc

SEQ ID NO: 146 - DNA, 20 bp, Zea mays
tccacagctttcttgaactt

SEQ ID NO: 147 - DNA, 20 bp, Zea mays
tcgcaccatcaagattcaaa

SEQ ID NO: 148 - DNA, 21 bp, Zea mays
tcgcttggtgcagatcgggac

SEQ ID NO: 149 - DNA, 21 bp, Zea mays
tcggaccaggcttcaatccct

SEQ ID NO: 150 - DNA, 19 bp, Zea mays
tcggaccaggcttcattcc

SEQ ID NO: 151 - DNA, 20 bp, Zea mays
tcggaccaggcttcattccc

SEQ ID NO: 152 - DNA, 21 bp, Zea mays
tcggaccaggcttcattcccc

SEQ ID NO: 153 - DNA, 21 bp, Zea mays
tcggaccaggcttcattcctc

SEQ ID NO: 154 - DNA, 22 bp, Zea mays
tgaagctgccagcatgatctgg

SEQ ID NO: 155 - DNA, 20 bp, Zea mays
tgacagaagagagtgagcac

SEQ ID NO: 156 - DNA, 21 bp, Zea mays
tgacagaagagagtgagcacg

SEQ ID NO: 157 - DNA, 21 bp, Zea mays
tgagccgtgccaatatcacaa

SEQ ID NO: 158 - DNA, 21 bp, Zea mays
tgagccgtgccaatatcacgt

SEQ ID NO: 159 - DNA, 21 bp, Zea mays
tgattgagccgtgccaatatc

SEQ ID NO: 160 - DNA, 18 bp, Zea mays
tgcagtctccttggctta

SEQ ID NO: 161 - DNA, 21 bp, Zea mays
tgcagttgttgtctcaagctt

SEQ ID NO: 162 - DNA, 19 bp, Zea mays
tgccaaaggagaattgccc

SEQ ID NO: 163 - DNA, 21 bp, Zea mays
tgccaaaggagagctgccctg

SEQ ID NO: 164 - DNA, 21 bp, Zea mays
tgccaaaggagagctgtcctg

SEQ ID NO: 165 - DNA, 21 bp, Zea mays
tgccaaaggagagttgccctg

SEQ ID NO: 166 - DNA, 20 bp, Zea mays
tgcctggctccctgtatgcc

SEQ ID NO: 167 - DNA, 20 bp, Zea mays
tgcctggctccctgtatggc

SEQ ID NO: 168 - DNA, 23 bp, Zea mays
tgctctctgctctcactgtcatc

SEQ ID NO: 169 - DNA, 21 bp, Zea mays
tggaaggggcatgcagaggag

SEQ ID NO: 170 - DNA, 21 bp, Zea mays
tggagaagaagggcacatgca

SEQ ID NO: 171 - DNA, 20 bp, Zea mays
tggagaagcagggcacgtgc

SEQ ID NO: 172 - DNA, 21 bp, Zea mays
tggagaagcagggcacgtgca

SEQ ID NO: 173 - DNA, 20 bp, Zea mays
tggcgctagaaggagggcac

SEQ ID NO: 174 - DNA, 20 bp, Zea mays
tggcgctagaaggagggcca

SEQ ID NO: 175 - DNA, 20 bp, Zea mays
tggcgctagaaggagggctc

SEQ ID NO: 176 - DNA, 19 bp, Zea mays
tgggagatgaaggagccat

SEQ ID NO: 177 - DNA, 19 bp, Zea mays
tgggagatgaaggagccgt

SEQ ID NO: 178 - DNA, 19 bp, Zea mays
tgggagatgaaggagcctt

SEQ ID NO: 179 - DNA, 21 bp, Zea mays
tgtgttctcaggtcgcccccg

SEQ ID NO: 180 - DNA, 21 bp, Zea mays
tgttggcacggttcaatcaaa

SEQ ID NO: 181 - DNA, 20 bp, Zea mays
ttagaagaagaagaagaaga

SEQ ID NO: 182 - DNA, 21 bp, Zea mays
ttagatgaccatcagcaaaca

SEQ ID NO: 183 - DNA, 22 bp, Zea mays
ttcagtttcctctaatatctca

SEQ ID NO: 184 - DNA, 22 bp, Zea mays
ttccacaggctttcttgaactg

SEQ ID NO: 185 - DNA, 22 bp, Zea mays
ttcctaatgcctcccattccta

SEQ ID NO: 186 - DNA, 22 bp, Zea mays
ttcctgatgcctcctattccta

SEQ ID NO: 187 - DNA, 22 bp, Zea mays
ttcctgatgcctctcattccta

SEQ ID NO: 188 - DNA, 22 bp, Zea mays
ttcctgatgtctcccattccta

SEQ ID NO: 189 - DNA, 21 bp, Zea mays
ttggactgaagggtgctccct

SEQ ID NO: 190 - DNA, 20 bp, Zea mays
ttggcattctgtccacctcc

SEQ ID NO: 191 - DNA, 21 bp, Zea mays
tttggagtgaagggagctctg

SEQ ID NO: 192 - DNA, 22 bp, Zea mays
tttggatctgctattttggtat

SEQ ID NO: 193 - DNA, 21 bp, Zea mays
tttggattgaagggagctcga

SEQ ID NO: 194 - DNA, 20 bp, Zea mays
tttggattgaagggagctct

SEQ ID NO: 195 - DNA, 21 bp, Zea mays
tttggattgaagggagctctg

SEQ ID NO: 196 - DNA, 22 bp, Zea mays
tttgttttcctctaatatctca

SEQ ID NO: 197 - DNA, 22 bp, Zea mays
tttgttttcctctaatatctta

SEQ ID NO: 198 - DNA, 21 bp, Zea mays
agaagagagagagtacagcct

SEQ ID NO: 199 - DNA, 20 bp, Glycine max
tgacagaagagagtgagcac

SEQ ID NO: 200 - DNA, 21 bp, Glycine max
tgacagaagagagagagcaca

SEQ ID NO: 201 - DNA, 21 bp, Glycine max
ttgacagaagatagagagcac

SEQ ID NO: 202 - DNA, 21 bp, Glycine max
ttgacagaagatagagagcac

SEQ ID NO: 203 - DNA, 21 bp, Glycine max
ttgacagaagatagagagcac

SEQ ID NO: 204 - DNA, 22 bp, Glycine max
ttgacagaagagagagagcaca

SEQ ID NO: 205 - DNA, 20 bp, Glycine max
acagaagatagagagcacag

SEQ ID NO: 206 - DNA, 20 bp, Glycine max
tgacagaagagagtgagcac

SEQ ID NO: 207 - DNA, 21 bp, Glycine max
ttgacagaagatagagagcac

SEQ ID NO: 208 - DNA, 21 bp, Glycine max
ttgacagaagatagagagcac

SEQ ID NO: 209 - DNA, 20 bp, Glycine max
tgacagaagagagtgagcac

SEQ ID NO: 210 - DNA, 20 bp, Glycine max
tgacagaagagagtgagcac

SEQ ID NO: 211 - DNA, 20 bp, Glycine max
tgacagaagagagtgagcac

SEQ ID NO: 212 - DNA, 20 bp, Glycine max
tgacagaagagagtgagcac

SEQ ID NO: 213 - DNA, 20 bp, Glycine max
tgacagaagagagtgagcac

SEQ ID NO: 214 - DNA, 20 bp, Glycine max
tgacagaagagagtgagcac

SEQ ID NO: 215 - DNA, 20 bp, Glycine max
tgacagaagagagtgagcac

SEQ ID NO: 216 - DNA, 20 bp, Glycine max
tgacagaagagagtgagcac

SEQ ID NO: 217 - DNA, 20 bp, Glycine max
tgacagaagagagtgagcac

SEQ ID NO: 218 - DNA, 21 bp, Glycine max
ttgacagaagatagagagcac

SEQ ID NO: 219 - DNA, 21 bp, Glycine max
ttgacagaagatagagagcac

SEQ ID NO: 220 - DNA, 21 bp, Glycine max
ttgacagaagagagtgagcac

SEQ ID NO: 221 - DNA, 21 bp, Glycine max
tttggattgaagggagctcta

SEQ ID NO: 222 - DNA, 21 bp, Glycine max
attggagtgaagggagctcca

SEQ ID NO: 223 - DNA, 21 bp, Glycine max
attggagtgaagggagctccg

SEQ ID NO: 224 - DNA, 21 bp, Glycine max
agctgcttagctatggatccc

SEQ ID NO: 225 - DNA, 21 bp, Glycine max
attggagtgaagggagctcct

SEQ ID NO: 226 - DNA, 19 bp, Glycine max
attggagtgaagggagctc

SEQ ID NO: 227 - DNA, 19 bp, Glycine max
attggagtgaagggagctc

SEQ ID NO: 228 - DNA, 21 bp, Glycine max
atttaagtgatgggagctccg

SEQ ID NO: 229 - DNA, 21 bp, Glycine max
tttggattgaagggagctcta

SEQ ID NO: 230 - DNA, 20 bp, Glycine max
gagctccttgaagtccaatt

SEQ ID NO: 231 - DNA, 21 bp, Glycine max
tgcctggctccctgtatgcca

SEQ ID NO: 232 - DNA, 21 bp, Glycine max
tgcctggctccctgtatgcca

SEQ ID NO: 233 - DNA, 21 bp, Glycine max
tgcctggctccctgtatgcca

SEQ ID NO: 234 - DNA, 21 bp, Glycine max
tgcctggctccctgtatgcca

SEQ ID NO: 235 - DNA, 21 bp, Glycine max
tgcctggctccctgtatgcca

SEQ ID NO: 236 - DNA, 21 bp, Glycine max
tgcctggctccctgtatgcca

SEQ ID NO: 237 - DNA, 20 bp, Glycine max
tcgataaacctctgcatcca

SEQ ID NO: 238 - DNA, 20 bp, Glycine max
tcgataaacctctgcatcca

SEQ ID NO: 239 - DNA, 20 bp, Glycine max
tcgataaacctctgcatcca

SEQ ID NO: 240 - DNA, 21 bp, Glycine max
tggagaagcagggcacgtgca

SEQ ID NO: 241 - DNA, 21 bp, Glycine max
tggagaagcagggcacgtgca

SEQ ID NO: 242 - DNA, 21 bp, Glycine max
tggagaagcagggcacgtgca

SEQ ID NO: 243 - DNA, 21 bp, Glycine max
tggagaagcagggcacgtgca

SEQ ID NO: 244 - DNA, 21 bp, Glycine max
tggagaagcagggcacgtgca

SEQ ID NO: 245 - DNA, 21 bp, Glycine max
tggagaagcagggcacgtgca

SEQ ID NO: 246 - DNA, 21 bp, Glycine max
tggagaagcagggcacgtgca

SEQ ID NO: 247 - DNA, 21 bp, Glycine max
tggagaagcagggcacgtgca

SEQ ID NO: 248 - DNA, 21 bp, Glycine max
tggagaagcagggcacgtgca

SEQ ID NO: 249 - DNA, 21 bp, Glycine max
tggagaagcagggcacgtgca

SEQ ID NO: 250 - DNA, 21 bp, Glycine max
tggagaagcagggcacgtgca

SEQ ID NO: 251 - DNA, 21 bp, Glycine max
tggagaagcagggcacgtgca

SEQ ID NO: 252 - DNA, 21 bp, Glycine max
tcggaccaggcttcattcccc

SEQ ID NO: 253 - DNA, 21 bp, Glycine max
tcggaccaggcttcattcccc

SEQ ID NO: 254 - DNA, 21 bp, Glycine max
tcggaccaggcttcattcccc

SEQ ID NO: 255 - DNA, 21 bp, Glycine max
tcggaccaggcttcattcccc

SEQ ID NO: 256 - DNA, 21 bp, Glycine max
tcggaccaggcttcattcccc

SEQ ID NO: 257 - DNA, 21 bp, Glycine max
tcggaccaggcttcattcccc

SEQ ID NO: 258 - DNA, 20 bp, Glycine max
tcggaccaggcttcattccc

SEQ ID NO: 259 - DNA, 21 bp, Glycine max
tcggaccaggcttcattcccc

SEQ ID NO: 260 - DNA, 20 bp, Glycine max
tcggaccaggcttcattccc

SEQ ID NO: 261 - DNA, 21 bp, Glycine max
tcggaccaggcttcattcccc

SEQ ID NO: 262 - DNA, 21 bp, Glycine max
tcggaccaggcttcattcccc

SEQ ID NO: 263 - DNA, 21 bp, Glycine max
tcggaccaggcttcattcccc

SEQ ID NO: 264 - DNA, 21 bp, Glycine max
tcggaccaggcttcattcccc

SEQ ID NO: 265 - DNA, 20 bp, Glycine max
tcggaccaggcttcattccc

SEQ ID NO: 266 - DNA, 21 bp, Glycine max
tcggaccaggcttcattcccc

SEQ ID NO: 267 - DNA, 21 bp, Glycine max
tcggaccaggcttcattcccc

SEQ ID NO: 268 - DNA, 20 bp, Glycine max
tctcggaccaggcttcattc

SEQ ID NO: 269 - DNA, 21 bp, Glycine max
tctcggaccaggcttcattcc

SEQ ID NO: 270 - DNA, 21 bp, Glycine max
tctcggaccaggcttcattcc

SEQ ID NO: 271 - DNA, 21 bp, Glycine max
tgaagctgccagcatgatcta

SEQ ID NO: 272 - DNA, 21 bp, Glycine max
tgaagctgccagcatgatcta

SEQ ID NO: 273 - DNA, 21 bp, Glycine max
tgaagctgccagcatgatctg

SEQ ID NO: 274 - DNA, 21 bp, Glycine max
tgaagctgccagcatgatcta

SEQ ID NO: 275 - DNA, 21 bp, Glycine max
tgaagctgccagcatgatctt

SEQ ID NO: 276 - DNA, 21 bp, Glycine max
tgaagctgccagcatgatctt

SEQ ID NO: 277 - DNA, 22 bp, Glycine max
tgaagctgccagcatgatctga

SEQ ID NO: 278 - DNA, 20 bp, Glycine max
tgaagctgccagcatgatct

SEQ ID NO: 279 - DNA, 21 bp, Glycine max
tcgcttggtgcaggtcgggaa

SEQ ID NO: 280 - DNA, 21 bp, Glycine max
tcgcttggtgcaggtcgggaa

SEQ ID NO: 281 - DNA, 21 bp, Glycine max
cagccaaggatgacttgccgg

SEQ ID NO: 282 - DNA, 21 bp, Glycine max
cagccaaggatgacttgccga

SEQ ID NO: 283 - DNA, 21 bp, Glycine max
aagccaaggatgacttgccga

SEQ ID NO: 284 - DNA, 23 bp, Glycine max
tgagccaaggatgacttgccggt

SEQ ID NO: 285 - DNA, 20 bp, Glycine max
agccaaggatgacttgccgg

SEQ ID NO: 286 - DNA, 21 bp, Glycine max
cagccaaggatgacttgccgg

SEQ ID NO: 287 - DNA, 19 bp, Glycine max
cagccaaggatgacttgcc

SEQ ID NO: 288 - DNA, 21 bp, Glycine max
tttcgacgagttgttcttggc

SEQ ID NO: 289 - DNA, 21 bp, Glycine max
tagccaagaatgacttgccgg

SEQ ID NO: 290 - DNA, 21 bp, Glycine max
cagccaagaatgacttgccgg

SEQ ID NO: 291 - DNA, 21 bp, Glycine max
cagccaagaatgacttgccgg

SEQ ID NO: 292 - DNA, 21 bp, Glycine max
cagccaaggatgacttgccgg

SEQ ID NO: 293 - DNA, 21 bp, Glycine max
cagccaagggtgatttgccgg

SEQ ID NO: 294 - DNA, 19 bp, Glycine max
cagccaaggatgacttgcc

SEQ ID NO: 295 - DNA, 21 bp, Glycine max
cagccaaggatgacttgccgg

SEQ ID NO: 296 - DNA, 21 bp, Glycine max
taattgagccgcgtcaatatc

SEQ ID NO: 297 - DNA, 21 bp, Glycine max
tgagccgtgccaatatcacga

SEQ ID NO: 298 - DNA, 21 bp, Glycine max
cgagccgaatcaatatcactc

SEQ ID NO: 299 - DNA, 23 bp, Glycine max
acggcgtgatattggtacggctc

SEQ ID NO: 300 - DNA, 21 bp, Glycine max
agatattggtgcggttcaatc

SEQ ID NO: 301 - DNA, 21 bp, Glycine max
tgattgagccgtgccaatatc

SEQ ID NO: 302 - DNA, 21 bp, Glycine max
tgattgagccgtgccaatatc

SEQ ID NO: 303 - DNA, 21 bp, Glycine max
tgattgagccgtgccaatatc

SEQ ID NO: 304 - DNA, 21 bp, Glycine max
tgattgagccgtgccaatatc

SEQ ID NO: 305 - DNA, 21 bp, Glycine max
ttgagccgcgccaatatcact

SEQ ID NO: 306 - DNA, 21 bp, Glycine max
ttgagccgcgccaatatcact

SEQ ID NO: 307 - DNA, 21 bp, Glycine max
tgattgagccgtgccaatatc

SEQ ID NO: 308 - DNA, 21 bp, Glycine max
tgattgagccgtgccaatatc

SEQ ID NO: 309 - DNA, 21 bp, Glycine max
tgattgagccgtgccaatatc

SEQ ID NO: 310 - DNA, 20 bp, Glycine max
ttgagccgcgtcaatatctt

SEQ ID NO: 311 - DNA, 20 bp, Glycine max
ttgagccgcgtcaatatctt

SEQ ID NO: 312 - DNA, 21 bp, Glycine max
cgagccgaatcaatatcactc

SEQ ID NO: 313 - DNA, 21 bp, Glycine max
tgattgagccatgtcaatatc

SEQ ID NO: 314 - DNA, 21 bp, Glycine max
ttgagccgtgccaatatcacg

SEQ ID NO: 315 - DNA, 21 bp, Glycine max
agaatcttgatgatgctgcat

SEQ ID NO: 316 - DNA, 21 bp, Glycine max
agaatcttgatgatgctgcat

SEQ ID NO: 317 - DNA, 21 bp, Glycine max
ggaatcttgatgatgctgcag

SEQ ID NO: 318 - DNA, 24 bp, Glycine max
ggaatcttgatgatgctgcagcag

SEQ ID NO: 319 - DNA, 24 bp, Glycine max
ggaatcttgatgatgctgcagcag

SEQ ID NO: 320 - DNA, 20 bp, Glycine max
agaatcttgatgatgctgca

SEQ ID NO: 321 - DNA, 20 bp, Glycine max
gcagcaccatcaagattcac

SEQ ID NO: 322 - DNA, 21 bp, Glycine max
agaatcttgatgatgctgcat

SEQ ID NO: 323 - DNA, 20 bp, Glycine max
gaatcttgatgatgctgcat

SEQ ID NO: 324 - DNA, 21 bp, Glycine max
agaatcttgatgatgctgcat

SEQ ID NO: 325 - DNA, 20 bp, Glycine max
gaatcttgatgatgctgcat

SEQ ID NO: 326 - DNA, 20 bp, Glycine max
gaatcttgatgatgctgcat

SEQ ID NO: 327 - DNA, 20 bp, Glycine max
ttggactgaagggagctccc

SEQ ID NO: 328 - DNA, 20 bp, Glycine max
ttggactgaagggagctccc

SEQ ID NO: 329 - DNA, 20 bp, Glycine max
ttggactgaaaggagctcct

SEQ ID NO: 330 - DNA, 20 bp, Glycine max
ttggactgaagggagctccc

SEQ ID NO: 331 - DNA, 21 bp, Glycine max
tggactgaagggagctccttc

SEQ ID NO: 332 - DNA, 23 bp, Glycine max
ttggactgaaggggagctccttc

SEQ ID NO: 333 - DNA, 22 bp, Glycine max
ttggactgaagggagctccctt

SEQ ID NO: 334 - DNA, 22 bp, Glycine max
ttggactgaagggagctccctt

SEQ ID NO: 335 - DNA, 21 bp, Glycine max
tggactgaagggagctccttc

SEQ ID NO: 336 - DNA, 22 bp, Glycine max
ttggactgaagggagctccctt

SEQ ID NO: 337 - DNA, 20 bp, Glycine max
ttggactgaagggagctccc

SEQ ID NO: 338 - DNA, 21 bp, Glycine max
aagctcaggagggatagcacc

SEQ ID NO: 339 - DNA, 21 bp, Glycine max
aagctcaggagggatagcgcc

SEQ ID NO: 340 - DNA, 20 bp, Glycine max
cgctatccatcctgagtttc

SEQ ID NO: 341 - DNA, 21 bp, Glycine max
aagctcaggagggatagcgcc

SEQ ID NO: 342 - DNA, 21 bp, Glycine max
aagctcaggagggatagcgcc

SEQ ID NO: 343 - DNA, 20 bp, Glycine max
agctcaggagggatagcgcc

SEQ ID NO: 344 - DNA, 20 bp, Glycine max
cgctatccatcctgagtttc

SEQ ID NO: 345 - DNA, 21 bp, Glycine max
tccaaagggatcgcattgatc

SEQ ID NO: 346 - DNA, 22 bp, Glycine max
tccaaagggatcgcattgatcc

SEQ ID NO: 347 - DNA, 22 bp, Glycine max
tccaaagggatcgcattgatcc

SEQ ID NO: 348 - DNA, 22 bp, Glycine max
tccaaagggatcgcattgatcc

SEQ ID NO: 349 - DNA, 22 bp, Glycine max
tccaaagggatcgcattgatcc

SEQ ID NO: 350 - DNA, 22 bp, Glycine max
tccaaagggatcgcattgatcc

SEQ ID NO: 351 - DNA, 22 bp, Glycine max
ttccaaagggatcgcattgatc

SEQ ID NO: 352 - DNA, 22 bp, Glycine max
ttccaaagggatcgcattgatc

SEQ ID NO: 353 - DNA, 22 bp, Glycine max
ttccaaagggatcgcattgatc

SEQ ID NO: 354 - DNA, 22 bp, Glycine max
ttccaaagggatcgcattgatc

SEQ ID NO: 355 - DNA, 21 bp, Glycine max
tccaaagggatcacattgatc

SEQ ID NO: 356 - DNA, 20 bp, Glycine max
agctctgttggctacacttt

SEQ ID NO: 357 - DNA, 20 bp, Glycine max
aggtgggcatactgtcaact

SEQ ID NO: 358 - DNA, 20 bp, Glycine max
ttggcattctgtccacctcc

SEQ ID NO: 359 - DNA, 21 bp, Glycine max
ctgaagtgtttgggggaactc

SEQ ID NO: 360 - DNA, 21 bp, Glycine max
ctgaagtgtttgggggaactc

SEQ ID NO: 361 - DNA, 21 bp, Glycine max
ctgaagtgtttgggggaactc

SEQ ID NO: 362 - DNA, 21 bp, Glycine max
atgaagtgtttgggagaactc

SEQ ID NO: 363 - DNA, 21 bp, Glycine max
atgaagtgtttgggggaactc

SEQ ID NO: 364 - DNA, 21 bp, Glycine max
atgaagtgtttgggggaactc

SEQ ID NO: 365 - DNA, 21 bp, Glycine max
atgaagtgtttgggggaactc

SEQ ID NO: 366 - DNA, 21 bp, Glycine max
atgaagtgtttgggggaactc

SEQ ID NO: 367 - DNA, 21 bp, Glycine max
atgaagtgtttgggggaactc

SEQ ID NO: 368 - DNA, 21 bp, Glycine max
ttccacagctttcttgaactg

SEQ ID NO: 369 - DNA, 21 bp, Glycine max
ttccacagctttcttgaactt

SEQ ID NO: 370 - DNA, 21 bp, Glycine max
ttccacagctttcttgaactt

SEQ ID NO: 371 - DNA, 24 bp, Glycine max
aagaaagctgtgggagaatatggc

SEQ ID NO: 372 - DNA, 22 bp, Glycine max
ttccacagctttcttgaactgt

SEQ ID NO: 373 - DNA, 20 bp, Glycine max
ttccacagctttcttgaact

SEQ ID NO: 374 - DNA, 20 bp, Glycine max
ttccacagctttcttgaact

SEQ ID NO: 375 - DNA, 21 bp, Glycine max
tcattgagtgcagcgttgatg

SEQ ID NO: 376 - DNA, 21 bp, Glycine max
tcattgagtgcagcgttgatg

SEQ ID NO: 377 - DNA, 21 bp, Glycine max
tgtgttctcaggtcacccctt

SEQ ID NO: 378 - DNA, 21 bp, Glycine max
tgtgttctcaggtcacccctt

SEQ ID NO: 379 - DNA, 21 bp, Glycine max
atgcactgcctcttccctggc

SEQ ID NO: 380 - DNA, 21 bp, Glycine max
atgcactgcctcttccctggc

SEQ ID NO: 381 - DNA, 20 bp, Glycine max
ctgggaacaggcagggcacg

SEQ ID NO: 382 - DNA, 21 bp, Glycine max
atgcactgcctcttccctggc

SEQ ID NO: 383 - DNA, 22 bp, Glycine max
agaatttgtgggaatgggctga

SEQ ID NO: 384 - DNA, 22 bp, Glycine max
tatggggggattgggaaggaat

SEQ ID NO: 385 - DNA, 23 bp, Glycine max
atttgtgggaatgggctgattgg

SEQ ID NO: 386 - DNA, 22 bp, Glycine max
tcttccctacacctcccatacc

SEQ ID NO: 387 - DNA, 22 bp, Glycine max
tatggggggattgggaaggaat

SEQ ID NO: 388 - DNA, 24 bp, Glycine max
tcttcccaattccgcccattccta

SEQ ID NO: 389 - DNA, 20 bp, Glycine max
tgcatttgcacctgcacttt

SEQ ID NO: 390 - DNA, 20 bp, Glycine max
tgcatttgcacctgcacttt

SEQ ID NO: 391 - DNA, 20 bp, Glycine max
tgcatttgcacctgcacttt

SEQ ID NO: 392 - DNA, 22 bp, Glycine max
tcttgctcaaatgagtattcca

SEQ ID NO: 393 - DNA, 22 bp, Glycine max
tctcattccatacatcgtctga

SEQ ID NO: 394 - DNA, 21 bp, Glycine max
tctcattccatacatcgtctg

SEQ ID NO: 395 - DNA, 23 bp, Glycine max
tctagaaagggaaatagcagttg

SEQ ID NO: 396 - DNA, 21 bp, Glycine max
tagaaaggggaatagcagttg

SEQ ID NO: 397 - DNA, 22 bp, Glycine max
ttaatcaaggaaatcacggtcg

SEQ ID NO: 398 - DNA, 21 bp, Glycine max
ttaatcaaggaaatcacggtt

SEQ ID NO: 399 - DNA, 23 bp, Glycine max
ttgttgttttacctattccaccc

SEQ ID NO: 400 - DNA, 24 bp, Glycine max
agggataggtaaaacaatgactgc

SEQ ID NO: 401 - DNA, 21 bp, Glycine max
tgttgttttacctattccacc

SEQ ID NO: 402 - DNA, 20 bp, Glycine max
aaccaggctctgataccatg

SEQ ID NO: 403 - DNA, 22 bp, Glycine max
taactgaaaattcttaaagtat

SEQ ID NO: 404 - DNA, 21 bp, Glycine max
taactggaaattcttaaagca

SEQ ID NO: 405 - DNA, 22 bp, Glycine max
taactgaacattcttagagcat

SEQ ID NO: 406 - DNA, 21 bp, Glycine max
tgagagaaagccatgacttac

SEQ ID NO: 407 - DNA, 21 bp, Glycine max
tgagagaaagccatgacttac

SEQ ID NO: 408 - DNA, 21 bp, Glycine max
tgagagaaagccatgacttac

SEQ ID NO: 409 - DNA, 21 bp, Glycine max
ttcatttttaaaataggcatt

SEQ ID NO: 410 - DNA, 21 bp, Glycine max
ttcatttttaaaatagacatt

SEQ ID NO: 411 - DNA, 22 bp, Glycine max
tcattttgcgtgcaatgatctg

SEQ ID NO: 412 - DNA, 22 bp, Glycine max
tcattttgcgtgcaatgatctg

SEQ ID NO: 413 - DNA, 21 bp, Glycine max
caaaagagcttatggcttgta

SEQ ID NO: 414 - DNA, 21 bp, Glycine max
caaaagagcttatgacttgta

SEQ ID NO: 415 - DNA, 21 bp, Glycine max
caaaagtacttgtggcttgta

SEQ ID NO: 416 - DNA, 24 bp, Glycine max
agtcttggtcaatgtcgttcgaaa

SEQ ID NO: 417 - DNA, 21 bp, Glycine max
tgtgttgtaaagtgaatatca

SEQ ID NO: 418 - DNA, 22 bp, Glycine max
taagtgttgcaaaatagtcatt

SEQ ID NO: 419 - DNA, 23 bp, Glycine max
tagaacatgatacatgacagtca

SEQ ID NO: 420 - DNA, 23 bp, Glycine max
gtgacagtcatcatttaataaga

SEQ ID NO: 421 - DNA, 23 bp, Glycine max
ttcaataagaacgtgacacgtga

SEQ ID NO: 422 - DNA, 22 bp, Glycine max
atcagaacatgacacgtgacaa

SEQ ID NO: 423 - DNA, 24 bp, Glycine max
caataagaacgtgacatatgacag

SEQ ID NO: 424 - DNA, 24 bp, Glycine max
caatcagaacatgacacatgacaa

SEQ ID NO: 425 - DNA, 24 bp, Glycine max
caatcagaacatgacacgtgacaa

SEQ ID NO: 426 - DNA, 24 bp, Glycine max
aacgtccaatcagaacgtgacatg

SEQ ID NO: 427 - DNA, 24 bp, Glycine max
aacgtgacacgtgacggtcaacat

SEQ ID NO: 428 - DNA, 24 bp, Glycine max
aagaacgtgacacatgacaatcaa

SEQ ID NO: 429 - DNA, 24 bp, Glycine max
aatcagaacatgacacatgacagt

SEQ ID NO: 430 - DNA, 24 bp, Glycine max
aatcagaacatgacacgtgatagt

SEQ ID NO: 431 - DNA, 24 bp, Glycine max
aatcagaacatgacatgtgacaat

SEQ ID NO: 432 - DNA, 24 bp, Glycine max
tcaatcagaacatgacacgtgaca

SEQ ID NO: 433 - DNA, 24 bp, Glycine max
tcatcgtccaatcagaatgtgaca

SEQ ID NO: 434 - DNA, 24 bp, Glycine max
atgttgttattggatgatgacggt

SEQ ID NO: 435 - DNA, 24 bp, Glycine max
attgaccaatcagaacatgacaca

SEQ ID NO: 436 - DNA, 24 bp, Glycine max
tgtcacatcctggttggacatgaa

SEQ ID NO: 437 - DNA, 20 bp, Glycine max
ctgttaatggaaaatgttga

SEQ ID NO: 438 - DNA, 20 bp, Glycine max
atgggataaatgtgagctca

SEQ ID NO: 439 - DNA, 21 bp, Glycine max
cgagtccgaggaaggaactcc

SEQ ID NO: 440 - DNA, 22 bp, Glycine max
ccggaagaggaaaattaagcaa

SEQ ID NO: 441 - DNA, 23 bp, Glycine max
ttaaaggaaacaattaatcgtta

SEQ ID NO: 442 - DNA, 23 bp, Glycine max
tcgtccatatgggaagacttgtc

SEQ ID NO: 443 - DNA, 23 bp, Glycine max
aacacgctaagcgagaggagctc

SEQ ID NO: 444 - DNA, 20 bp, Glycine max
tattttgggtaaatagtcat

SEQ ID NO: 445 - DNA, 19 bp, Glycine max
cttgtttgtggtgatgtct

SEQ ID NO: 446 - DNA, 21 bp, Glycine max
aagcagagacaaatgtgttta

SEQ ID NO: 447 - DNA, 21 bp, Glycine max
caaacctccgtagcctgtatc

SEQ ID NO: 448 - DNA, 21 bp, Glycine max
ttaatgtgttgtgtttgtcgg

SEQ ID NO: 449 - DNA, 21 bp, Glycine max
ttaatgtgttgtgtttgtgag

SEQ ID NO: 450 - DNA, 20 bp, Glycine max
tgcgagtgtcttcgcctctg

SEQ ID NO: 451 - DNA, 21 bp, Glycine max
gtccttgggatgcagattacg

SEQ ID NO: 452 - DNA, 21 bp, Glycine max
tcaaagggagttgtaggggaa

SEQ ID NO: 453 - DNA, 21 bp, Glycine max
tgagaccaaatgagcagctga

SEQ ID NO: 454 - DNA, 22 bp, Glycine max
tgcagagatagggacgcgctta

SEQ ID NO: 455 - DNA, 24 bp, Glycine max
tgtgttgaaagtttaacatgacgg

SEQ ID NO: 456 - DNA, 24 bp, Glycine max
aatcgacttagaatgtaggatggt

SEQ ID NO: 457 - DNA, 24 bp, Glycine max
aaaaaacttacggatcaagttgat

SEQ ID NO: 458 - DNA, 24 bp, Glycine max
tcttacagatcaagttgattcgga

SEQ ID NO: 459 - DNA, 24 bp, Glycine max
aagtagacattctaagacgttgct

SEQ ID NO: 460 - DNA, 22 bp, Glycine max
taagacggaacttacaaagatt

SEQ ID NO: 461 - DNA, 24 bp, Glycine max
gaaagaccaaacgagaagctgcat

SEQ ID NO: 462 - DNA, 24 bp, Glycine max
aagcttcttacggatcaagttgat

SEQ ID NO: 463 - DNA, 22 bp, Glycine max
aaacttgtaagatggtgacatt

SEQ ID NO: 464 - DNA, 24 bp, Glycine max
tattggctagagataagacaaaga

SEQ ID NO: 465 - DNA, 22 bp, Glycine max
tcaaatgattttgtgtcgttgg

SEQ ID NO: 466 - DNA, 22 bp, Glycine max
attgggattcagttggagttgg

SEQ ID NO: 467 - DNA, 24 bp, Glycine max
atttctaggacatactacgacggt

SEQ ID NO: 468 - DNA, 24 bp, Glycine max
caagtcgtagccggtgttattact

SEQ ID NO: 469 - DNA, 22 bp, Glycine max
caattggatcggtccaaccggc

SEQ ID NO: 470 - DNA, 22 bp, Glycine max
cactgttgtgctgggtgtacca

SEQ ID NO: 471 - DNA, 24 bp, Glycine max
caggactgtcttagaaagccaggc

SEQ ID NO: 472 - DNA, 24 bp, Glycine max
cagtcgtgtgattgtacggttcat

SEQ ID NO: 473 - DNA, 22 bp, Glycine max
cagtgcatgactatatcgccag

SEQ ID NO: 474 - DNA, 24 bp, Glycine max
aacgaagtgactctaacatcggtt

SEQ ID NO: 475 - DNA, 24 bp, Glycine max
aacgcgtgatatgttaacatcggt

SEQ ID NO: 476 - DNA, 24 bp, Glycine max
cagttgacgtacgtacggattgac

SEQ ID NO: 477 - DNA, 24 bp, Glycine max
ccggaagagacttacggatcaact

SEQ ID NO: 478 - DNA, 22 bp, Glycine max
ccttaggacagacgtcatgtag

SEQ ID NO: 479 - DNA, 22 bp, Glycine max
cgattaccagaaggcttattag

SEQ ID NO: 480 - DNA, 24 bp, Glycine max
cgcgagatcgcacggaagaaggtt

SEQ ID NO: 481 - DNA, 24 bp, Glycine max
taacaacagcggaagaaccttctt

SEQ ID NO: 482 - DNA, 24 bp, Glycine max
aagaacttcttccgcgagatcgca

SEQ ID NO: 483 - DNA, 22 bp, Glycine max
ctacttagtagagatttgttgg

SEQ ID NO: 484 - DNA, 22 bp, Glycine max
ctgaaccctagcgaagtaaatc

SEQ ID NO: 485 - DNA, 24 bp, Glycine max
aagacggtacttacctcagtaaca

SEQ ID NO: 486 - DNA, 24 bp, Glycine max
aaggacggtacttacgtaagcaac

SEQ ID NO: 487 - DNA, 24 bp, Glycine max
ggatcaagctgatccggaagtgga

SEQ ID NO: 488 - DNA, 24 bp, Glycine max
agtagactcgtccgattttgcgta

SEQ ID NO: 489 - DNA, 24 bp, Glycine max
aagtgatgacatgacaagcgaagt

SEQ ID NO: 490 - DNA, 24 bp, Glycine max
aagtgatgacgtggtagacggagt

SEQ ID NO: 491 - DNA, 24 bp, Glycine max
gacgtgacagacggaatatcacat

SEQ ID NO: 492 - DNA, 24 bp, Glycine max
taaaatcgtgacatgtgacggtca

SEQ ID NO: 493 - DNA, 24 bp, Glycine max
aagttgacgtacgtacggattgac

SEQ ID NO: 494 - DNA, 22 bp, Glycine max
taagacggtcgtgatgtcagca

SEQ ID NO: 495 - DNA, 22 bp, Glycine max
tactttcaaagacgttgttgag

SEQ ID NO: 496 - DNA, 24 bp, Glycine max
taccactagtggtcgcgcctggca

SEQ ID NO: 497 - DNA, 22 bp, Glycine max
tacgcaggagagatgacgctgt

SEQ ID NO: 498 - DNA, 24 bp, Glycine max
tacgtcatcgctgaatggaagacg

SEQ ID NO: 499 - DNA, 24 bp, Glycine max
ataggactgtcttagaatggtgta

SEQ ID NO: 500 - DNA, 24 bp, Glycine max
tagaactgtcttagaatgtgctac

SEQ ID NO: 501 - DNA, 24 bp, Glycine max
tagagtgtatactgtgagaggcct

SEQ ID NO: 502 - DNA, 24 bp, Glycine max
cggattgttgatccgtatgtgcat

SEQ ID NO: 503 - DNA, 22 bp, Glycine max
tatggtcatacggattgttgat

SEQ ID NO: 504 - DNA, 24 bp, Glycine max
tatgtgacggtaaacggtgacaag

SEQ ID NO: 505 - DNA, 22 bp, Glycine max
tatgttaactgatttcatggat

SEQ ID NO: 506 - DNA, 24 bp, Glycine max
tattggatctcagttgaaccggtc

SEQ ID NO: 507 - DNA, 24 bp, Glycine max
aatcagacactgcattcaaagacg

SEQ ID NO: 508 - DNA, 24 bp, Glycine max
aatcgatgtagaaaagtgattggt

SEQ ID NO: 509 - DNA, 24 bp, Glycine max
tcgaaggttctggagaggactgca

SEQ ID NO: 510 - DNA, 24 bp, Glycine max
aacaagacgtgatgacgtgacact

SEQ ID NO: 511 - DNA, 24 bp, Glycine max
aaggtgtgatggcatgacactctg

SEQ ID NO: 512 - DNA, 24 bp, Glycine max
agcgtgatgacgtgacactccgtc

SEQ ID NO: 513 - DNA, 24 bp, Glycine max
atgtcactgattaggcatgatgat

SEQ ID NO: 514 - DNA, 21 bp, Glycine max
tgttagtgataaggcgtgatg

SEQ ID NO: 515 - DNA, 24 bp, Glycine max
aatcttagggaccaaattgacagc

SEQ ID NO: 516 - DNA, 24 bp, Glycine max
tcggtcggaccgatccaatcggaa

SEQ ID NO: 517 - DNA, 24 bp, Glycine max
tcgtactcgtcgggtatcgggtat

SEQ ID NO: 518 - DNA, 24 bp, Glycine max
tctcggcaaagaactaagaagaag

SEQ ID NO: 519 - DNA, 22 bp, Glycine max
tctgcgaaaatgtgatttcgga

SEQ ID NO: 520 - DNA, 24 bp, Glycine max
tgagaaaaggacggcagaaaagcc

SEQ ID NO: 521 - DNA, 24 bp, Glycine max
ttgaaaagggacagcagagaagcc

SEQ ID NO: 522 - DNA, 24 bp, Glycine max
aatggactaaagagaaaggggccg

SEQ ID NO: 523 - DNA, 22 bp, Glycine max
tggataggagtatgggcttgag

SEQ ID NO: 524 - DNA, 24 bp, Glycine max
tgtagtttctaagacgatgctgac

SEQ ID NO: 525 - DNA, 22 bp, Glycine max
tgtcaaagatgtggcgaatact

SEQ ID NO: 526 - DNA, 24 bp, Glycine max
tgtcagcggagtgagaagacgaaa

SEQ ID NO: 527 - DNA, 22 bp, Glycine max
ttaacgaaaaaggactaacgac

SEQ ID NO: 528 - DNA, 24 bp, Glycine max
ttcggaaaaattctggaagacgtc

SEQ ID NO: 529 - DNA, 24 bp, Glycine max
acaacgtctttgaaagtaggcatt

SEQ ID NO: 530 - DNA, 24 bp, Glycine max
acatattatgggtctcagacggac

SEQ ID NO: 531 - DNA, 24 bp, Glycine max
acggacaccgaacacgacacggac

SEQ ID NO: 532 - DNA, 24 bp, Glycine max
attcgtggaagactggcggatcaa

SEQ ID NO: 533 - DNA, 24 bp, Glycine max
attctaagacggttatctgggacc

SEQ ID NO: 534 - DNA, 24 bp, Glycine max
attgattctgagagaaccggtgta

SEQ ID NO: 535 - DNA, 22 bp, Glycine max
cagaggaagcagcacttgtacc

SEQ ID NO: 536 - DNA, 24 bp, Glycine max
taacaacattggatgagggttgga

SEQ ID NO: 537 - DNA, 22 bp, Glycine max
taacaagtgggtttgttgactg

SEQ ID NO: 538 - DNA, 24 bp, Glycine max
tatgttgatccgtatgagtcgtac

SEQ ID NO: 539 - DNA, 22 bp, Glycine max
ttattgtaactaatttgtcggt

SEQ ID NO: 540 - DNA, 21 bp, Glycine max
agtggcgtagatccccacaac

SEQ ID NO: 541 - DNA, 20 bp, Glycine max
aagagaattgtaagtcactg

SEQ ID NO: 542 - DNA, 20 bp, Glycine max
taagagaattgtaagtcact

SEQ ID NO: 543 - DNA, 20 bp, Glycine max
agctgctgactcgttggctc

SEQ ID NO: 544 - DNA, 21 bp, Glycine max
aagttgtgatgagaatcaatg

SEQ ID NO: 545 - DNA, 21 bp, Glycine max
ttgattctcatcacaacatgg

SEQ ID NO: 546 - DNA, 20 bp, Glycine max
acgggtcgctctcacctagg

SEQ ID NO: 547 - DNA, 21 bp, Glycine max
acgggtcgctctcacctggag

SEQ ID NO: 548 - DNA, 21 bp, Glycine max
ttatagtctgacatctggaat

SEQ ID NO: 549 - DNA, 19 bp, Glycine max
tgcgagaggcacggggttc

SEQ ID NO: 550 - DNA, 22 bp, Glycine max
atttaaaattattgatttgtca

SEQ ID NO: 551 - DNA, 19 bp, Glycine max
gtcgttgtagtatagtggt

SEQ ID NO: 552 - DNA, 22 bp, Glycine max
cctgtcgtaggagagatgacgc

SEQ ID NO: 553 - DNA, 22 bp, Glycine max
ttgccgattccacccattccta

SEQ ID NO: 554 - DNA, 22 bp, Glycine max
ttgccgattccacccattccta

SEQ ID NO: 555 - DNA, 21 bp, Zea mays
tgtgctctctctcttctgtca

SEQ ID NO: 556 - DNA, 499 bp, Zea mays
ttgtagttggctttagctatcgtcatccccactgggtgctatcttatctcctatttcaatgggaacccacccaatggtcatgttggagacaacacctgttatggtcctttgaccatctcgtggtgactgtagttgatgtcatattcggatatatatgtaaaaggacctgcatagcaattgttagacctttgggaaagcaaaagcgataaagagatctcagatagatattgtgttctttcagacggtggttcctattcctatcaatcggttaatccatcccacatgggaggatttgtggtaagcttagtcagcaaacctctggtggtccctgaaggcctgaactttatcgggagagctgctgtagcaatccccgaagccgtgtgccgtttatcttgtcgggcatactttatttgccgagttgcagttattaagcacagggaggtgcggaatgcacttcgggcgtcgaagatatttttgctcagattccttgcgtctcaat

SEQ ID NO: 557 - DNA, 17 bp, Zea mays
gttatggtcctttgacc

SEQ ID NO: 558 - DNA, 200 bp, Zea mays
caaaggtctaacaattgctatgcaggtccttttacatatatatccgaatatgacatcaactacagtcaccacgagatggtcaaaggaccatgacagaagagagagagcacaaacaggtgttgtctccaacatgaccattgggtgggttcccattgaaataggagataagatagcacccagtggggatgacgatagctaaa

SEQ ID NO: 559 - DNA, 21 bp, Zea mays
aggctgtactctctctcttct

SEQ ID NO: 560 - DNA, 421 bp, Zea mays
cgacgcacactggccgcgggcgcgagacattgtccggccgtgtcacgcacgcccgcgtcctcctcctcctccgccgccgcgtaacgcacggccacgacgtgtccgtggtcgtaagtgctgtgtctgtgtgtaccaataaataagccccgttttgcttcgtccagaacggtccagtgctacgtgtagtgtatctgtgttgtgatttgcgaattggattattgtgggtcgtctcgtcgaggtctctcgggtgtcgggtgggtctgatgcgatccatcagcgtcgtgtccgaataaaagccacgccgatgcgccggctgacgggcatctggatgtgtgatttctgaacaagatttgcttaatttcacttgcttaatttccgctgcgtcactccttgagtccttggagccggcctctcgtctctc

SEQ ID NO: 561 - DNA, 17 bp, Zea mays
ccgcgtaacgcacggcc

SEQ ID NO: 562 - DNA, 19 bp, Zea mays
tggtcgtaagtgctgtgtc

SEQ ID NO: 563 - DNA, 200 bp, Zea mays
ggtgagacgccgcgcacgcagcgcagcttccgccgctgacgacgcacactggccgcgggcgcgagacattgtccggccgtgtcacgcaggctgtactctctctcttctgcgccgcgtaacgcacggccacgacgtgtccgtggtcgtaagtgctgtgtctgtgtgtaccaataaataagccccgttttgcttcgtccaga

SEQ ID NO: 564 - DNA, 4350 bp, Zea mays
ctccgttgtttatagtgtattattgttttgtgatgagctttgcttaaacaagctacttggaagcagtcctaagctgagacaatgttcttccatttccgcggctatatcttttagctcgcattattccgttattcgagagcagttgggggagatgtggcctcaagtggcgggatgccattgacctcgatccatccccctcaacatgtgcgtccgcagcctagctatatacaccatctcgacctccaggccccatcatcaccccagcacccaccccacctgcccacttcttcttcttcttcctcctctcgtctccttgcgttctccatcgcattgcatctgtagcgcgcagttctccctggtctttcgcatccgtgtctgcccattctgccgctcctagttaaacgagagaggctgttcttgtgtgagagagagctggacaccgtctccccacccattacgccgtagccaaacctggagaaaaacctgtgaagcatgtaactccagtccctgtccaaaatccaaacccaagccccccaaaaaaaacaaaaaaaaacaccttcaagatcgttctccaaaagctagcaccggtaatcagcgtcgaggcgtaagtcggtaggctggatacgcccccactaattccttagcacaaaatcaacaaaagcctccggatcatgtaagtttcgccaccatcatctttgctcgccgcccagctaaaaaaaacaaaaaaaattaagaaacccagcagcatgcatcgtcattagtagccatcagttgtagtctatcgatcgtcctgggagccatcatcccagcattatcgtagcattacctcagcaaaaaccatcaaatcatgtaattagtgtgctcccgcgtcctatttctgctgcactgccggcagtcgaaaaaaaccaaaaaaaaaagattgtctttgagagtaagtgagagagcaagcaggttgctagccgaccaggttgccagctagccatggagccggggtcgacgcccccgaacgggtcggcgccggcgacgcccggcacgccggcgccgctgttctccagcggcgggccgcgggtggactcgctgtcgtacgagcgcaagtcgatgccgcggtgcaagtgcctgccgctgccggcggtggaggggtggggcgtggcgacgcacacctgcgtcgtcgagatccccgcgccggacgtctcgctcacccgcaaggtacgccaccgccaccgtcccgtccacatgcgcgtgcacatgcacgctcgcatttctttctgctccttcttccttccttcctgagttccgtgatttttttttctgagtgcgcggtttatttttcatatttccacacgtctgatgatcttttagcattgtttttttgtgtgtttgtgtattgtgctattaattgttacttgagggtacagacatgcatcctcgcgaattttataccgtgtaggtcaagggctcaaagctaacgttacaacagtcttgtgtctgaaagcctgtagcgacacagccctgacagagtgacagtgctagtactttaagaaataaaaaaaaattgtaggcgtgatcgatgtcataagtttttgactgttgacaattgatcccttacagtacatgatcacctgatcatttctgtgatcttatatatactgctcgatcagtttggctctttgcatcgttcttgaaatctctcgatgttgcatgctgacgagagacatttggagcttgaagcagcaaatacaagtaggcaggcttgctagcatcgctgggtgtgatgggctgatcgttctccacgccgcctgaagccagcctgcttcacgggcgctgctgtgacctgtgagcctgcaagtacacacccaggtggctggaacttttggcttggcacaccatcgtgtcccccctaagagcatctccaaaagctcaccagaagtttcccctaaatctatttttttagaaaaaacacaaaaacatgtctccaacagttcccctaaagcgctaccaactttttcatagcccttaaaacttcctcttttgtagctacaaacgaggggttttttgggctccccagaaacaaactgtcgctttaagggatctgttggagaaataattaaaatctaccctacttattatttagatgtcccttaaaactgattttgaggagtcgttttatggagagctcttggagatgctccaaccttcattcggcttgttttctgtgttgttctccaggtccagggtaaccgctcccgggaaataaacgactcgcttgtttgaatttgcattgggttgaccaatttcgaattgctggatttgggttcgcgcgctgatcccggggaaaaaagtccaaaacaacgtaagaacaaatcaattatattgatccgtagtagagagctgaggtcttgttcccaaaaacatttattttaacaatggtattggttttggcctacaattaaagtagactttcgaatattgtttttattagtaggccatgaatagctagagtgtactgtcaaaaaagtggcgttctgaaaacgtgacaataaattacgtctgctccatgtacttagcttgtaaaagctcatgcatggggaagaatcgtgcacgtgaaccagatcgggtagcctgtcacgttcctcagccttgaatcgaaactacaccgggtctagttaattatatattgtcgataagagacaacgtacagtgtttttggaacaaagaaaaatggtacccccagctccaattatttcatgataaagtacggctcttggacatgacacggcaaaagggggcgacagtacgtcccaaactcgagtttgatcgaggcgtccgtgactggagtggttgcatacttgcatgcgttgttttgtctctctctccctctctgtgtcgctgaaatattggtcacgtccatcccaatctcatctcaaatctgtccatgggctgacaagaccccgcgcgctgaaagcatttgcgtggtggctgctgcacgcagctgggcgcggagttcgtgggcacgttcatcctcatcttcttcgcgacggcggcgccgatcgtgaaccagaagtacggcggcgcgatcagcccgttcggcaacgcggcgtgcgcggggctggcggtggcgaccgtcatcctgtcgacggggcacatctccggggcgcacctgaacccgtcgctcaccatcgccttcgcggcgctgcgccacttcccctggctgcaggtgcccgcgtacgtggccgtccaggcgctggcatccgtctgcgccgccttcgcgctcaagggcgtcttccacccgttcctctccggcggcgtcaccgtgcccgacgccaccatctccaccgcccaggcgttcttcaccgagttcatcatctccttcaacctcctcttcgtcgtcaccgccgtcgccaccgacacccgcgcagtacgcgttctttctctctctctctctctctctctttcagtgcattgacagcatgcatattgccatcgatgatcgattcagctctgaatttctgctgcccctgcccctgccctgcccttcgttcttgcaggtgggtgaactcgccgggatcgcggtgggagcggccgtaacgctgaacatcctcgtcgccgggtaagtctccttgctaccttatatgtgtttgtagtagcacgtgataaggtgatcatatcttcgtacgtgcacgcatgtggttggttgagctgacctgatgtgagcgcgtgtctccgggccgggcaggccgacgacgggcgggtccatgaacccggtgaggacgctggggccggccgtggcggcggggaactaccggcagctctggatctacctgctggccccgacgctgggcgcgttggcgggggccagcgtgtacacggcggtgaagctcagggacgagaacggtgagacgccgcgcacgcagcgcagcttccgccgctgacgacgcacactggccgcgggcgcgagacattgtccggccgtgtcacgcacgcccgcgtcctcctcctcctccgccgccgcgtaacgcacggccacgacgtgtccgtggtcgtaagtgctgtgtctgtgtgtaccaataaataagccccgttttgcttcgtccagaacggtccagtgctacgtgtagtgtatctgtgttgtgatttgcgaattggattattgtgggtcgtctcgtcgaggtctctcgggtgtcgggtgggtctgatgcgatccatcagcgtcgtgtccgaataaaagccacgccgatgcgccggctgacgggcatctggatgtgtgatttctgaacaagatttgcttaatttcacttgcttaatttccgctgcgtcactccttgagtccttggagccggcctctcgtctctc

SEQ ID NO: 565 - DNA, 8082 bp, Zea mays
cgctttgctttgtcgcgacgcgagcctgcgcgttgcgttcgtggcgtcggggagggggggggatggcaagcgccggtttgctgtattggtcacgtgatgcggctgcgcaacggccggggccagggcggtgccgccgcggcgacgggatcgcgcgtggttggtgtgacggacgggtgggggcgcaccgagcgcatgccgcctccatgaggattgaggaggccctcccgtccggatgcatcacacatgccactggcttggctggctgggacgtgatcccctgcaccggctacgtgttcttccttctcaccgagaagatgctgacaatgaaaacggcggcctcgtcacggctccgacgcgatccgtgggcgggcgggcgcgtccctcgccggagctggcgccgcgtgtcgccttctctggaacgccacgtgcctgcgtgcgcgcgcgcgcgcgagacgtcctagtccccagtgatttcgcttggaaattttttttatccgaatcttgagggtctggaggagcttcatggctaccgagtgcccgtcagcccgtggtatttttaggtgtgaacggagcccggttctcgtcggatcgccagctttaataagcacgaggtagtaggtgcgaagaggagaacggccttaactaactgctctcctcacgctgccaggaatgagcttgacgacggtgctagctggcacgaccttattaattttttattactatattactagaaaacgattgatcacggcgaaaattaagcactcgcctcatgacccgatcctccgaagccccttcgcatcacggcgctttgcggtgcttgtctttgacttcttcttttttttaaaaaaaaactcgcaatgggtagtacacacgatagccgcagtctttaaaagttgattactcttctcaatctgacctgacgtttggctcaagtcgaactcgagccgggaaaaaataaatatattagttaacaactaaaccctcctgtaaattaagcctaagtacctaggatacgtccctcctgcctctaactacttttttattactatctttattatcgatagaatctagggtgtgtttgtttagcgggttgacgcgggttggaacagattcaacacataacaactcgtgtttggtttaaaaggctcgcgggttagattggttccgggtcgaaatacgtcttgaaaatatgggttctaaggattcctaaaaacaagcgaaccatttcgacccacttcctctctaccctactgtcacccatgtcttatccactcgagaccagcacagggcgtgccgccactgtcgagggaccccgccgacctaccctccgacgttaatcccctgcctgccactagtcgtcggtcccccactggcctacccgccagcatgctggcctccgccagcgccgtcagactccctgaccggcttgttgcgcctcccaccgtcagactcctcggctggcatgccctccgatgtcgtggtgttctccgataggcacaacttgcgagccttgctagtgctgtcgtcgaacaggtaaaggtcagtgctcgtcatcgcctagacaagcctgctagtcatgtcaaatgttaaaaattcactgaaatgtatcttagtgttgttgggatgttcctttaactataactgaaatgtaacagtgagacctcatcactaaaaaattatgacttggttaataatggctttaatttatgtatatattgtgcttattgatttctcaaatattaaaatagtgtcgtttcatttattttgaatgttgtcatcttattgaattgaattgaagatatatcattttgggtaatatgaccagtaaggttactatattaagtttatccctcatcatgaaataaataagagatggtgtagctactatgagttctatgtctatctagtgtccatgccggacatgtttcgttgcatgcttaatagtagcgccgagagcgttccctcctcttctcgcaagcgtgcctgccaactttgaaaaaaaaaatatcttagatatacctttgctctttgctttgagcgcttgtattttgtccttctgtttcttctttaccgagttaagtgcttaactagcatttgtggcaatcaccatcgatcatctgatctatggctgcatctttcttcaatcttggatgtggacttgacctagcccacgcacaacgacacctgcagagtaactgttagcccactaattatctactaccaaacgcaaggttttcaaaatctcccctgtatatacatatatggctttcaatcccgcaaatagatacgttcattcataggagtacgcacgtgtgccgtgcacgagggtctcggtttataccgtcttcttttttttttgaagaagaaaaaaaataaacttgtcctaagcaggacgtacggtagcacctactgccaataagatatatattgctgctggtaaaatattttataatgtttatgatcttatcattgaaaatttcgtgtgaagtttttaaatttaaaattcaaattttataaacggtcttggatgtagaaactattaaaataaaacttttagatctcaaaaagttatgcaactttatagttggtcacattttcaaatgaactcatttagtgccttaaataatcaaattactctcgatttgttatagtacatgggaaatggaaacgtaatataaatataattggtgtaatagtgtagtgctatagaagggtatgcgcgagagagaggttgcgagttcgaatctcaccatttacaaaacatataagtttgattcaaaataatagtgaaaaatgatagggcaacgggtaagggtagggttggagagttgttcctagaatttaaaaaatgttttgctgttttttttttaatttttttgattcttaatttgccgagtgtttttctttgccgagtgctttttgacactcggcaaagtctttgccgagtgtccgaaaaaaacactcggcaaagaaccctttgccgatgaaatctttgtcgagtgttaagcctttgtcgagtgtaaaatagtctttgccgagtgtcttagacactcggcaaagaacgtgattccggtagtgaatagatacgttcattcataggagtacgcacgtgtgccgtgcaggagggtctcggtttacaccatgttctttttttagaagaaaaaaattaaacttgtcctaagcaggacgtacggtagcacctactgccaataagatatatattgctgctggtaaaatattttataatgtttatggctttataatgtttaaacttgtcctaatagctgacacatgacagactctaacctttcagtttccttgaaaagcagtaggccagcagccacaccccaggcgacagatggcgcgatcgtccacatggtggaggtggatagcagcaatccaggcttttccagtttggttccaaggcggcctccgtgtttttaattttacacacacatatatacatcctcctctcgttttcttcggccacttcttttttcttcttcttcttctttttgttgcacacatatatgtattccagggaaatttcccttggtgtgtgccatatacatgcatgtggaggagacggggatggagagctgtgagctagtcccctttgtactacagaaagaaagtgcgtgctgcccttatttctggaatgaaaaggcagaatgggaagagggggaaaaaaggggatagatagatgtggaggttgagggagacccacaccaccatataaacccccctcttccaaactagtcacacatggcacaagcacacagctcatcagttcttcttcgtcgtcttggctagctgcctagctagctagctagttcctcactcctctccgagaaccccctttttcatctctctctctctctctcggtctcgcccagctcctttcgatgttatcgtctctgctggcctcgcaactcgtcgagagagactcgtgaaaaagaatccagatatagctagagagaccgagagggcagctagcatgtcgatgagcttcttgagcatggtggaggcggagctgccgccggggttccggttccacccgagggacgacgagctcatctgcgactacctcgcgcccaagctcggcgccaagcccggcttctccggctgccgcccgcccatggtcgacgtcgacctcaacaaggtcgagccatgggatctccccggtgagtacgtacgtccacgcaagtagctagataaatcttttttttaatttgctagcgctcctttctttttctacgaccttcttctcagctaataaaagctagctagctgtgcgtcttccttgattaattcctccatgatacttgcaccaagttgatgcatatatgcgcacggagaggagaaagatgcagagaaatcacacacgtcaaatttccctagcttgctcgacacaacacgatccttaaatctgtccctgtttattatattctagtccacgatcaaaactcacagctttttccgtttttttaaataaaacacagcctcttaatttctgctgttttttttaaaaaaatcatgattttccagaataaagctatttgggatcgatcctctgttgcttcagcgtcaagagaaacaccacacgagtccccggccaaatgtgctgttcatacactaaactgctgcacacacatcacaccactctcctacatacttttatttttcgtcagcaagctagctaggatattaatatcgacgttttgctacctaaacctttagaaataattcatgatcgatctcgttttcatgcctatatatgatcatttccttgtcttcctcttcactcggtatatgttttctatacctcttctttttgttaccatgaacttgacttgttgatgacgcctccccaacaaatgaatagttatgaccagctgtattttaagtcggaggtcaaactaaaattaagcagtaggtatacacacaccattaattaattgaaaaacacatcgatttaatttgttttccgttcgtcggctgtaattatccaatttgtacacgatggtttctttgtttgtttctgaaaccttctcgcgcgcgcggtggtataatatcaacgtcacgatcagagtacgcgtgtacacgtactgcgctctcgatatgcacaagaccacactaacaaagctagccaagcgtccgtcgtttggacgagctttttgccaatctaaaaagaagagtaaatttaatttgaatcatttggggtggttcatctaaattcaccgcgaggaacgacaaccctctgctatatggtatatctctcgatttgtttaagctggtgaagccgtcctgctgcctgccaaatagatagactctagcaactactgagttttaaaaagagaaacaaaattaaagaatgacaaccttttacggccaaaccccttttaacgtctggccaacgaatcttctcttcgacctgttttagcttgtgtccaaaatgctttccgtcgtagccctgcgttcgttactagataccgctcaccatttgatcttgattatgaaaacattgaccttatatatatatatatatatatatagccaagtattttttaatctaatatggaataagagaaaaactaatgtgcgtgcaactatcagaagcaaaattttctgtttttttcttctaacattgttgggtaaattagaattcaaaattttatgtatcggtatactatatattataataccctccagctcagattcaaatccgttttagataattaatagattcatacaacacttaatggtgtgtgtacatgttttatacacgtgtctagattcattatcatctatttgaatatagtagacataaaaatcaagatcaaaaccgaatactactttagcacggagtgagtaaaagtataagtataatattttttggggatatttgggatttttttaattttgattacacgacgattatattatattgtacggccggggaagcgttgattgtgcacagactgcatgccgtacaaactgcaagaataatcgtacatgtacatgcatgctgatgaactgaaagatcttgagctcgtggccctcctaatttgttaaataaaaatgcacatcggaaagcaacaaactgcaaatcacgccttacgcatgagatctcagattcccagcacgctctctctctctctctctgtctgcacatcacgtcacgtgtccctcccctcttgtgaattgatgcccggatcagcacactactggtagcagaaggtcccaaaactaagctatgcatgggcgggctacatacatacaccacccccatgctatttctagcagagtggctccaaacctcagcatccacagtacaggctacagcgtgtggttgtgatggtgaccacaagaatcctactacatgcatgctgatgatgtgctgcagtgaacactcactcactgcccatgaacagcgtctcctacctgccagccagtgccaagaacaatgaatgaatgattaactgctggcttcgcttgctgaaccattcccctcctcgtgtgcgcatgatgggcatgcagtggcggcgtcggtggggccgcgggagtggtacttcttcagcctcaaggaccgcaagtacgcgacggggcagcggacgaaccgggccacggtgtccgggtactggaaggcgacggggaaggaccgaccagtggtggcggcgcggcgaggcgcgctggtggggatgcgcaagacgctcgtgttctaccaggggagggcgcccaagggcaggaagacggagtgggtgatgcacgagtacaggatggagccagctgctcctcttcttgatcaccaaccctcctcatccaactccaaggtaaaatcggtcgatccattattcgatgaaacgacaagttaagactccattaattcgacgaactgaccgggggtgttttaactgtttcgttgtcgtccatcgatccttctcctgtcatcatgtcaccagcaggatgaagattgggtgctgtgcagagtcatctgcaagaagaaactggcagcaggaggccgcgcaggagggggcagctcgaggagcctggtcgccagcaacggcggccgcgagaccgcgccagccaccccgccgccgccgccgctgccacctcgcatggacacggacgccaccctagcacagctccaggccgccatgcacgccaccgccggcgcgctcgagcaggtgccctgcttctccagcttcaacaacaacactgccagctctagagctgctgccgcagcagcagcagcgcagccatgctacctgcccagcatggccacaggcggcagccacggcacgacgagctactacctagaccacgcgatgctgccgcctgagctgggtggctgcttcgatcctctccacggcgacaagaagctgctcaaggcggtgctgggccagctcggcggcgacgcggtggcgccgggcctgagcctgcagcacgagatggccgcgggcgctgtcgtcgcttcatccgcttggatgaatcacttctaggggacattagttcagcagcgaacctaagtatgtgattggttgcgtatattatgggaaatacatacatatatacataattgtgtggtgaaacatttgtgtgtgaggcaaggagtagagcatgcgtcatttttttggttgctgccgatcgatttggacgaaggccatgaggcaaggagtagaactctgtaatataatgatggctgctcaagtgtgtgtacacattcagatgcagaggtcgtatgtcatgagcgatgatcaggagaatttcagattggttattttactgtgcctgaactcaaccccaagatcacacttgacaccactcactcactcactcactcagagacacagtgcacattcatagaatcatcatggtaaacagacagaagacagaacagtggctgccaaacccaatcaatcatgccatttatgcaggaaagtaggcatgacagtcacatccgagcatctcttctttaccacaccatctttctttttccgatgtgtattgttgcgatgacaatcatgctaaccaagcagtcagctgagtgagggctcctgggcactggctttccactgcact

SEQ ID NO: 566 - DNA, 23 bp, Zea mays
agcgaacctaagtatgtgattgg